(19) United States
(12) Patent Application Publication
(10) Pub. No.: US 2026/0351536 A1
(43) Pub. Date: Nov. 5, 2026

(54) PROTEOFORM IMAGING AND CHARACTERIZATION MASS SPECTROMETRY

(71) Applicant: Northwestern University, Evanston, IL (US)

(72) Inventors: Neil L. Kelleher, Evanston, IL (US); John P. McGee, Evanston, IL (US); Pei Su, Evanston, IL (US)

(21) Appl. No.: 19/483,051

(22) PCT Filed: May 10, 2024
(86) PCT No.: PCT/US2024/028952

§ 371 Date: Nov. 10, 2025

Related U.S. Application Data
(60) Provisional application No. 63/501,368, filed on May 10, 2023.

(51) Int. Cl. H01J 49/00

(52) U.S. Cl. CPC H01J 49/0004; H01J 49/0036

(57) ABSTRACT

Disclosed herein is a method for imaging or identification of proteoforms from a tissue sample. The method includes spatially surveying a first portion of the tissue sample to obtain survey data with a mass spectrometry apparatus, selecting from the survey data one or more proteoform targets and a mass-to-charge (m/z) isolation window for targeted fragmentation for each of the one or more proteoform target with a processor, generating a spatial acquisition sequence for a data acquisition survey of a second portion of the tissue sample with a processor and, spatially acquiring mass spectrometry data from the second portion of the tissue sample with the mass spectrometry apparatus executing the spatial acquisition sequence.

### CROSS REFERENCE TO RELATED APPLICATIONS

This application claims benefit of priority to U.S. Appl. Ser. No. 63/501,368, filed May 10, 2023, the contents of which is incorporated in its entirety.

### STATEMENT REGARDING FEDERALLY SPONSORED RESEARCH

This invention was made with government support under grant numbers 5674 Amnd 2//HU00012120002 awarded by Department of Defense and DA018310, GM108569, CA246635, and CA060553 awarded by the National Institutes of Health. The government has certain rights in the invention.

### FIELD OF THE INVENTION

The disclosed technology is generally directed to mass spectrometry. More particularly the technology is directed to tissue imaging and identification mass spectrometry.

### BACKGROUND OF THE INVENTION

A proteoform is a protein form of a gene product where the protein may contain one or more post-translational modifications (PTMs) and/or sequence variations. Proteoforms are key effectors of biological function. In addition to the molecular profile of endogenous proteoforms, complex phenotypes manifest as proteome heterogeneity across functional tissue in time and space. Mass spectrometry (MS) imaging can profile proteins with moderate spatial resolution without the need for affinity reagents or antibody labels. However, proteome complexity presents a grand challenge for “top-down” MS of intact proteoforms in tissue. When using electrospray for tissue desorption, each proteoform generates a distribution of charge states, resulting in congested spectra in the mass-to-charge (m/z) domain. As a result, only a few of the most abundant proteoforms can be targeted in traditional data acquisition schemes.

Recently, tissue imaging with proteoform-level information has been demonstrated. Due to the wide dynamic range, spectral complexity and spatial heterogeneity of the proteome, the approach has been limited to only target the most detectable proteoforms in tissue. More specifically, due to the limited pulses of ions from sampling tissue voxels versus the ion requirements for top-down fragmentation, protein identification is biased to high abundance and low molecular weight proteoforms (<20 kDa). Therefore, there is a need in the field for extended and streamlined spatial mapping and molecular identification of proteoform signatures.

### BRIEF SUMMARY OF THE INVENTION

Disclosed herein are methods and systems for imaging or identification of proteoforms from a tissue sample. The method comprising spatially surveying a first portion of the tissue sample to obtain survey data with a mass spectrometry apparatus; selecting from the survey data one or more proteoform targets and a mass-to-charge (m/z) isolation window for targeted fragmentation for each of the one or more proteoform target with a processor; generating a spatial acquisition sequence for a data acquisition survey of a second portion of the tissue sample with a processor and; spatially acquiring mass spectrometry data from the second portion of the tissue sample with the mass spectrometry apparatus executing the spatial acquisition sequence. The survey data may be obtained by proteoform imaging mass spectrometry. The mass spectrometry data is obtained by charge detection mass spectrometry, optionally wherein the charge detection mass spectrometry is individual ion mass spectrometry.

Another aspect of the technology provides for systems for imaging or identification of proteoforms from a tissue sample. The system comprising a mass spectrometry apparatus and a processor configured to perform any of the methods described herein. The processor may comprise instructions for selecting from survey data one or more proteoform targets and a mass-to-charge (m/z) isolation window for targeted fragmentation for each of the one or more proteoform targets and generating a spatial acquisition sequence for a data acquisition survey of a second portion of the tissue sample. The mass spectrometry apparatus can be configured to spatially acquire mass spectrometry data from a second portion of a tissue sample by executing the spatial acquisition sequence.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows logic and performance metrics of AutoPiMS (“Proteoform imaging Mass Spectrometry”) proteoform identification directly from ovarian cancer tissue. Panel a) shows a survey line scan and associated individual ion mass spectra detecting multiply-charged proteoform ions under denaturing conditions. Selected charge states of proteoforms are automatically targeted at optimized locations in a subsequent line scan for top-down fragmentation and database search. Panel b) shows a survey spectrum in the 17-50 kDa range with proteoform identification by the AutoPiMS workflow. The inset shows a zoomed view of the spectral region highlighted. In this region, Inventors detected phosphorylated proteoforms of heat shock protein beta-1 (HSPB1, UniProt accession: P04792) and a coding polymorphism that created two proteoforms of glutathione S-transferase P (GSTP1, UniProt accession: P09211). The inset shows gene ontology analysis of 73 MS²-identified proteoforms. Panel c) shows a bar plot showing the difference in abundance of 25 specific proteoforms in the survey line scan (dots) versus when they were fragmented in the subsequent identification scans (bars). Panel d) shows raw ion counts of the 25 proteoforms in the survey scan when spatial pixels are assigned to the 25 targets randomly (violin plots) versus algorithm-optimized (dots) in subsequent MS² fragmentation scans. Panel e) shows MS¹ survey and MS² spectra along with graphical fragment maps of N-terminal acetylated vimentin (UniProt accession: P08670) and N-terminal acetylated keratin type II cytoskeletal 8 (UniProt accession: P05787) identified by AutoPiMS. Theoretical isotopic distributions of the two proteoforms are overlayed. The step plots show the spatial distributions of the two proteoforms along the survey line and the locations where they were targeted.

FIG. 2 shows application of AutoPiMS to ovarian cancer tissue, including PiMS imaging, label-free quantitation, and automated MS² identification (all scale bars are 500 μm). Panel a) shows regions of interest used for label-free quantitation and imaging. Dashed line in the image depicts an MS² line scan. Panel b) shows a volcano plot generated from label-free quantitation of 552 proteoforms using the ion counts from 472 pixels in tumor and stroma regions. PiMS images of 10 proteoforms significantly enriched in tumor and 7 proteoforms in stroma region are correlated to their quantitation outcome in the volcano plot by dashed lines. Panel c) shows reproducibility of quantitation for the 17 differentially detected proteoforms highlighted in b). Panel d) shows principal component analysis of 472 region-of-interest pixels (using the ion counts of 552 proteoforms as dimensions) showing clear differentiation of tumor vs. stroma pixels (red and blue, respectively). Panel e) shows examples of the identification of highly-similar proteoforms, tropomyosin alpha-1 chain isoform 9 (UniProt accession: P09493-9), tropomyosin alpha-1 chain isoform 3 (UniProt accession: P09493-3), and tropomyosin beta chain isoform 2 (UniProt accession: P07951-2). Panel f) shows differential spatial localization of three proteoforms of CRIP1 (UniProt accession: P50238) with Arg68me0, me1 and me2; The imaged area highlighted using a dashed box on the histology imagecontains tumor, stroma, and vasculature regions. PiMS images of CRIP1 proteoforms are shown. The graphical fragment maps obtained from automated MS² characterize unmethylated CRIP1 and localize the mono- and di-methylations of CRIP1. A PiMS image of N-terminally acetylated vimentin (UniProt accession: P08670) serves as a marker for stromal and vascularized tumor regions. Merged image of vimentin and CRIP1 Arg68me0 show co-localization in tumor vascular regions.

FIG. 3 provides panel (a) shows m/z- and panel (b) shows mass-domain spectra of Pierce intact protein mixture. Panels (c) shows output of the algorithm for m/z isolation window selections for all six constituent proteins. The chart contains information for each protein: average mass, selected m/z isolation window, charge state, total ion count of the charge state, other species co-isolated in the selected m/z isolation window, and the relative abundance of the target protein in the selected m/z isolation window. The reconstructed m/z spectra of the six proteins and selected m/z isolation windows are shown in the panel. Panel (d) shows graphical fragment maps of the six proteins from their corresponding targeted fragmentation experiments using the algorithm's recommendations. Labels correspond to fragments identified using averaged composite spectrum and I²MS, respectively. Grey squares indicate disulfide bond locations (C) or deamidation (N).

FIG. 4 shows a full mass spectrum of the survey line scan on the high-grade serous ovarian cancer tissue containing 113 proteoforms with greater than 1% relative abundance ranging from 4-67 kDa.

FIG. 5 shows reconstruction of m/z spectra from a PiMS experiment using the top 5 targets, the top 50 targets, all charge assigned ions, and the averaged spectrum from the corresponding RAW file showing the complexity of the spectrum increases as more information is included.

FIG. 6 shows, in panel (a), a number of detected charge states of the 113 proteoforms in Table 1 plotted against average mass; panel (b) shows a heatmap of the m/z and average masses of the 113 proteoforms.

FIG. 7 shows reconstructed m/z spectra of automated PiMS-selected spatial pixels for the proteoforms of masses 19801 Da in panel (a), 17880 Da in panel (b), and 35921 Da in panel (c). The panels show the RAW spectra in full range and the corresponding automated PiMS-selected m/z isolation windows for the target. Inset % RA indicates the total relative abundances of the targets in the survey.

FIG. 8 shows a heatmap of spatial relative abundances of 79-targeted proteoforms. Each row represents the spatial relative abundances of a proteoform. The proteoforms (y-axis from top to bottom) are organized according to the spatial targeting sequence (topmost and bottommost proteoforms are targeted in the leftmost and rightmost pixel, respectively). The bar shows the relative abundances for the pixels. Upon automated MS² algorithm optimization, all the proteoform targets were assigned with a spatial pixel with a relatively high abundance (diagonal pixels are illuminated).

FIG. 9 shows GO analysis of the 73 MS²-identified proteoforms in a panel (a), showing “Cellular responses to stress” is the most enriched pathway. Protein-protein interaction (PPI) network analysis results of the 73 proteoforms are shown in panel (b).

FIG. 10 shows Total ion count distributions of the tumor and stroma pixels in the region-of-interest sampling (Rep #1 corresponds to the data shown in FIG. 2d, and Rep #2 and #3 are technical replicates from two adjacent tissue sections).

FIG. 11 shows unsupervised classification of tumor and stroma pixels in the two technical replicates (a and b) using principal component analysis in adjacent tissue sections.

FIG. 12 shows graphical fragment maps and MS² spectra of TPM1 isoforms shown in FIG. 2e (TPM1-9 (a) and TPM1-3 (b)).

FIG. 13 shows graphical fragment maps and MS² spectra of CRIP1 proteoforms (Arg68me0 (a), Arg68me1 (b), Arg68me2 (c)) shown in FIG. 2f.

FIG. 14 shows extended information of the human high-grade serous ovarian cancer tissue sections used in this study.

FIG. 15 shows estimation of the spatial resolution of the imaging experiment. The spatial resolution is defined by the distance between 20% and 80% relative abundance of a sharp change in a spatial line profile of CRIP1 Arg68me0 in the imaged region corresponding to a boundary of the tissue.

### DETAILED DESCRIPTION OF THE INVENTION

To streamline and extend proteome coverage, Inventors developed technology for unattended proteoform identification in spatial proteomics and applied it to human ovarian cancer tissues. The technology augments proteoform imaging MS (PiMS) with an engine for molecular identification and, in conjunction with label-free quantitation, advances the field of molecular histology.

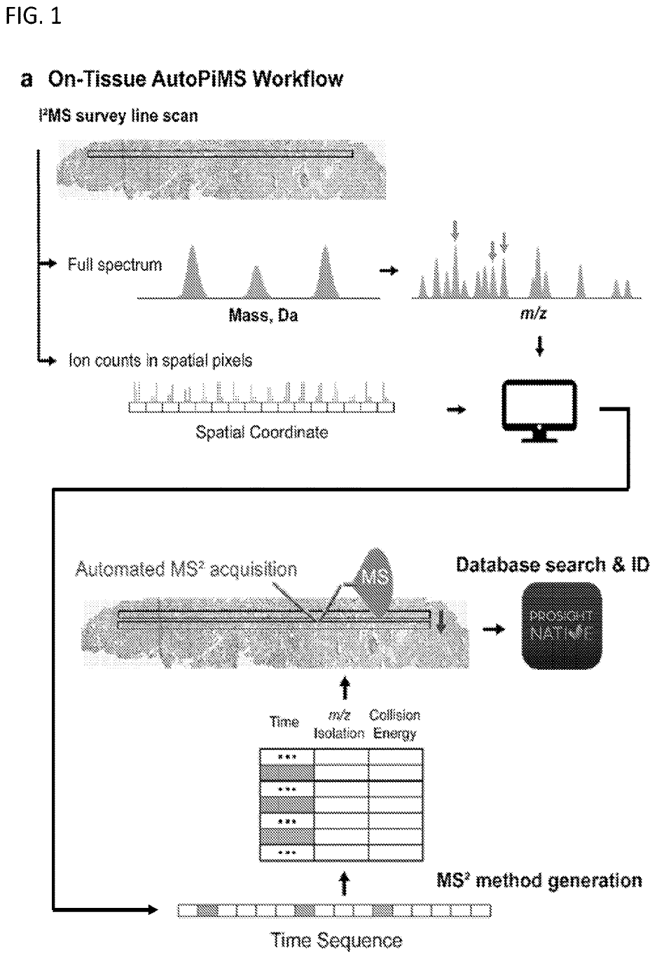

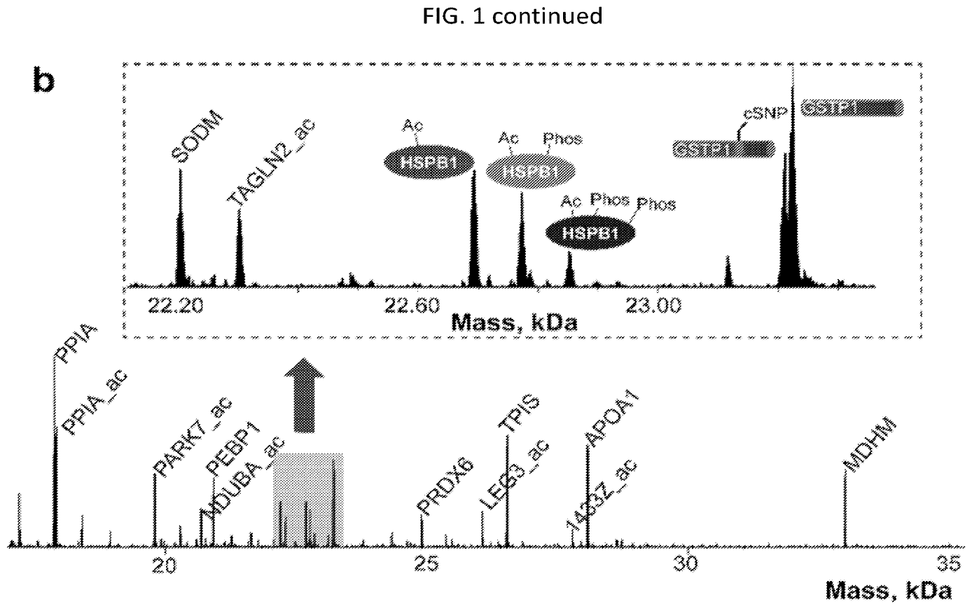

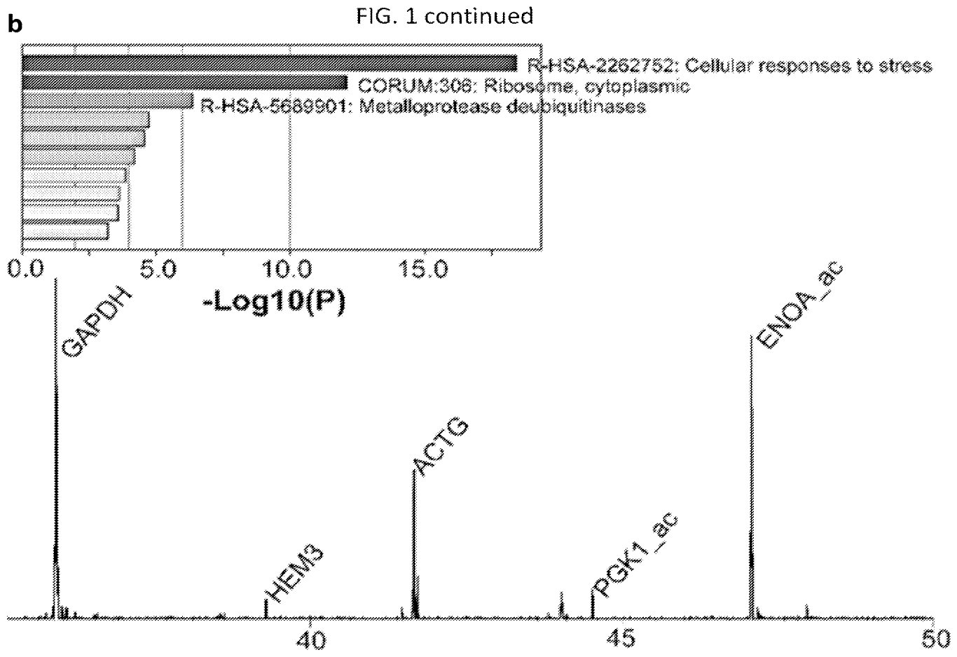

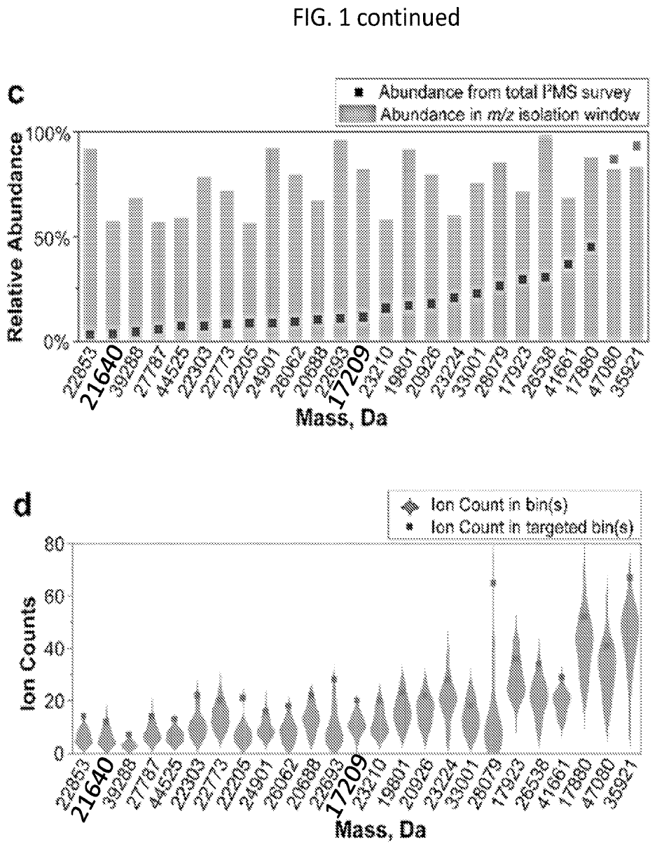

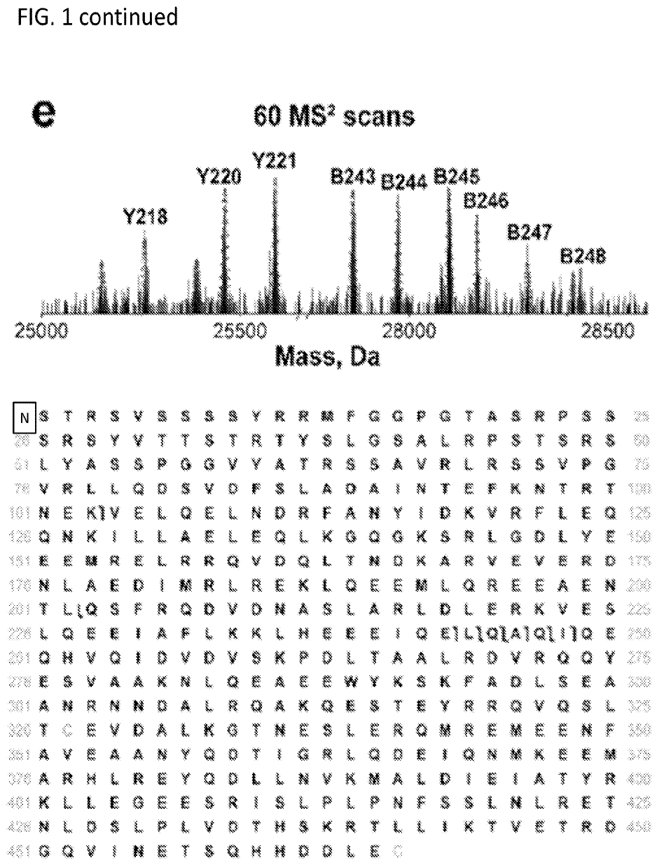

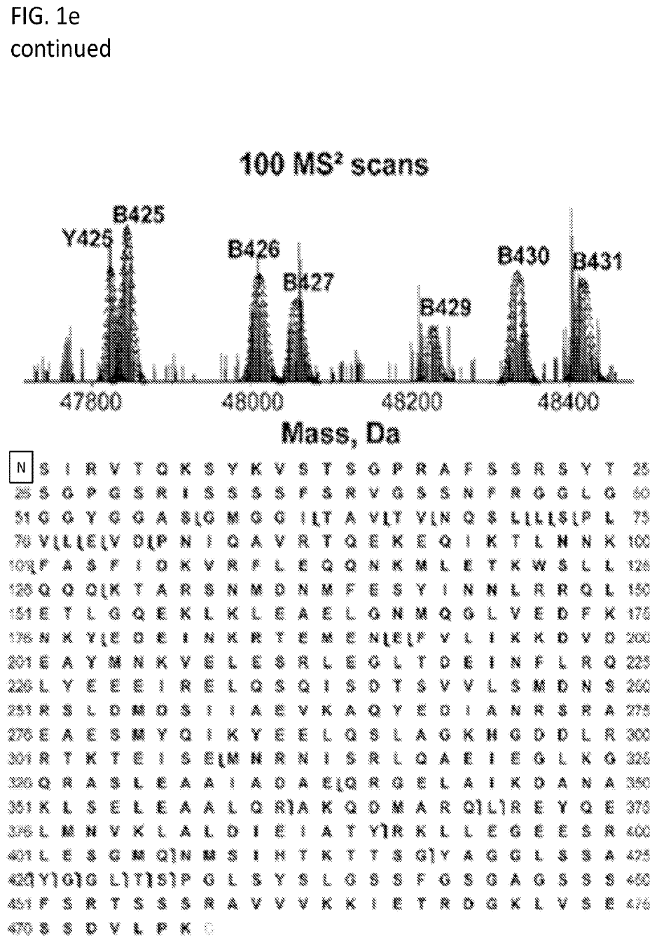

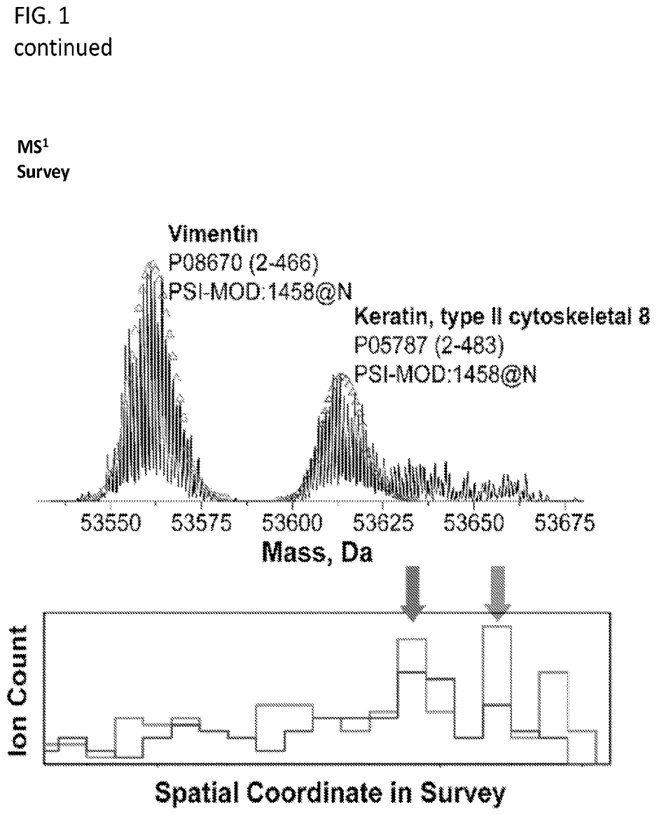

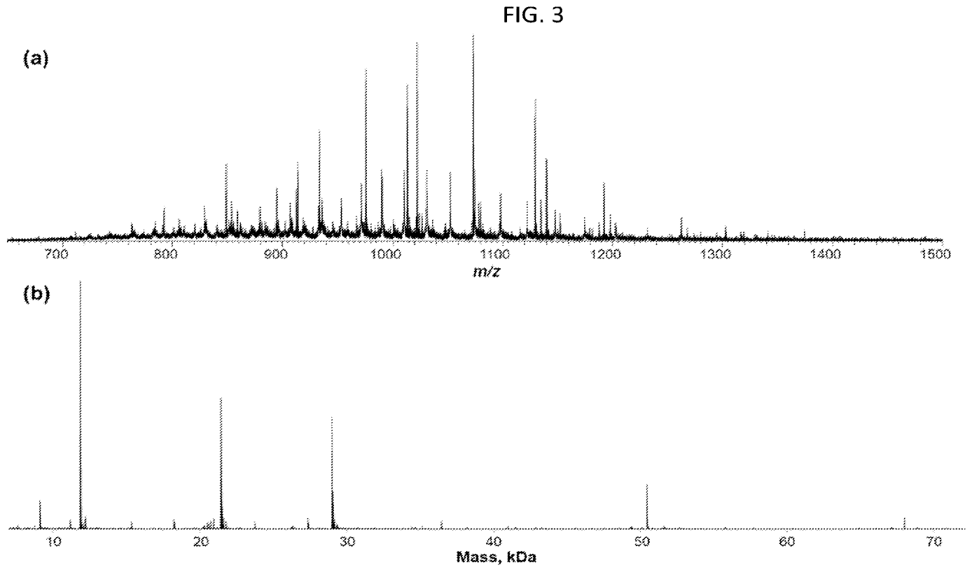

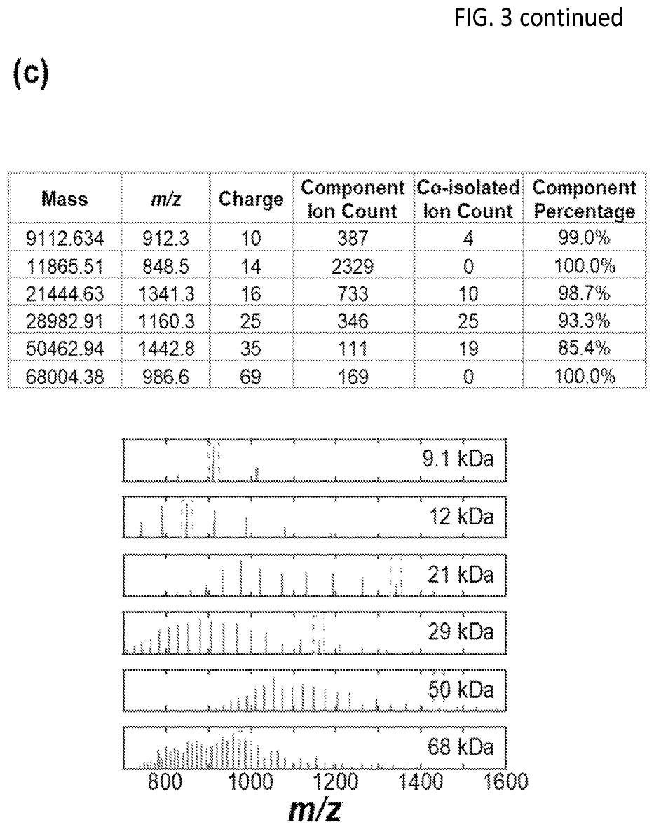

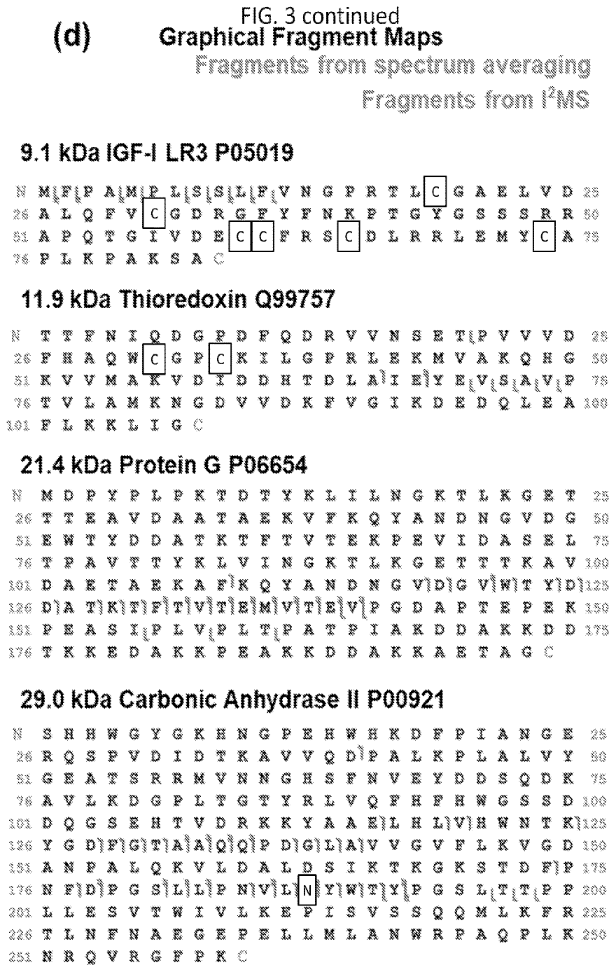

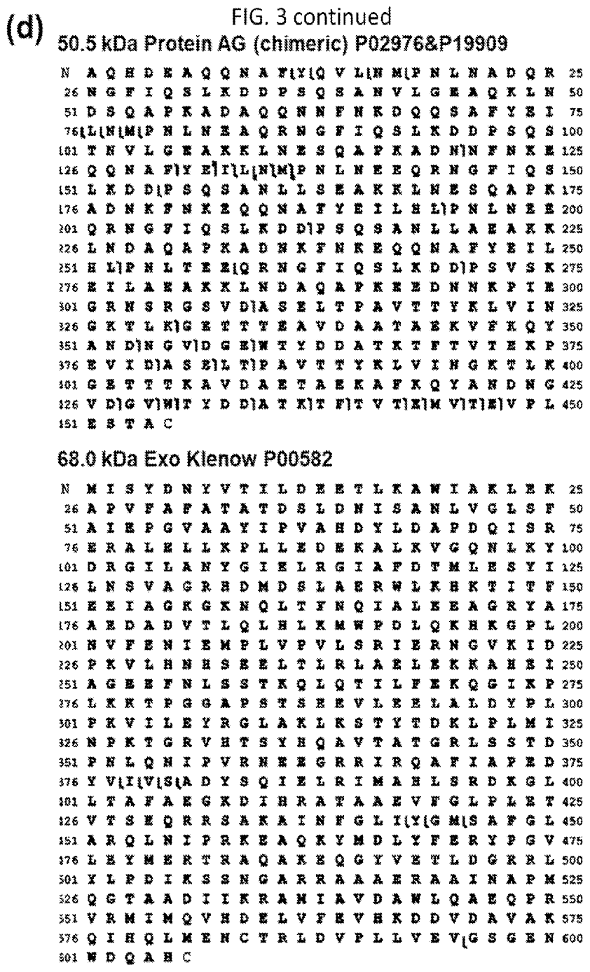

The disclosed technology achieves unattended identification of proteoforms using a spatially-aware, data-dependent acquisition strategy. The logic of the workflow is depicted in FIG. 1a: step 1) a PiMS line scan to obtain proteoform absolute mass, charge, and spatial distribution; step 2) target selection using an algorithm that finds optimal isolation windows for targeted fragmentation; and step 3) automated creation of a tandem MS (MS²) acquisition method within the contexts of m/z and tissue location. MS² fragmentation is then performed by running the PiMS probe across a fresh line parallel to the survey line scan but offset or shifted, e.g., by ~200 μm, 180 μm, 160 μm, 140 μm, 120 μm, 100 μm, 80 μm, 60 μm, 40 μm, or 20 μm (FIG. 1a). MS² fragmentation data may be acquired in either conventional ensemble mode for lower molecular weight proteoform targets, e.g., less than 17 kDa, 16 kDa, 15 kDa, 14 kDa, 13 kDa, 12 kDa, 11 kDa, or 10 kDa, or I²MS mode for larger proteoforms, e.g., greater than 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa, or 60 kDa. For ensemble mode, the probe may be scanned at 3 μm/s and MS² data acquired at 14 Hertz (Hz). For I²MS the probe scan rate may proceed at 2 μm/s, and the spectral acquisition rate may be acquired at a rate of 1 Hz. The workflow may include a search engine for proteoform identification compatible with the individual ion, tandem mass spectrometry (MS²) data type (FIG. 1a). Direct infusion of a standard mixture of six intact proteins was employed for proof-of-concept, and the workflow readily characterized all six components at the MS² level (FIG. 3).

PiMS combines nanospray desorption electrospray ionization mass spectrometry imaging (nano-DESI MSI) with data acquisition and processing for individual ions for the proteoform-selective imaging of biological tissues. Nano-DESI generates multiply charged protein ions, which is advantageous for their structural characterization using MS² directly on the tissue. PiMS utilizes nano-DESI line scans on tissue, during which proteoforms are sampled as multiply charged ions distributed across multiple charge states. Instead of unresolved signals from overlapping charge states of protein mixtures typically observed for ensembles of ions, PiMS provides information of individual protein ions in the mass domain even when their charge states overlap in the mass-to-charge domain. Enabled by direct charge assignment to individual ion signals, PiMS generates mass-domain information of the individual proteoforms with resolution of peaks in each pixel of the imaging data. This allows for confident assignment of proteoform masses with better than 2-ppm accuracy at 1σ in each pixel of the imaging data. Beyond proteoform-specific images, molecular identification may be achieved using direct top-down MS/MS off the tissue and supplemented by database searching from known intact proteoform masses.

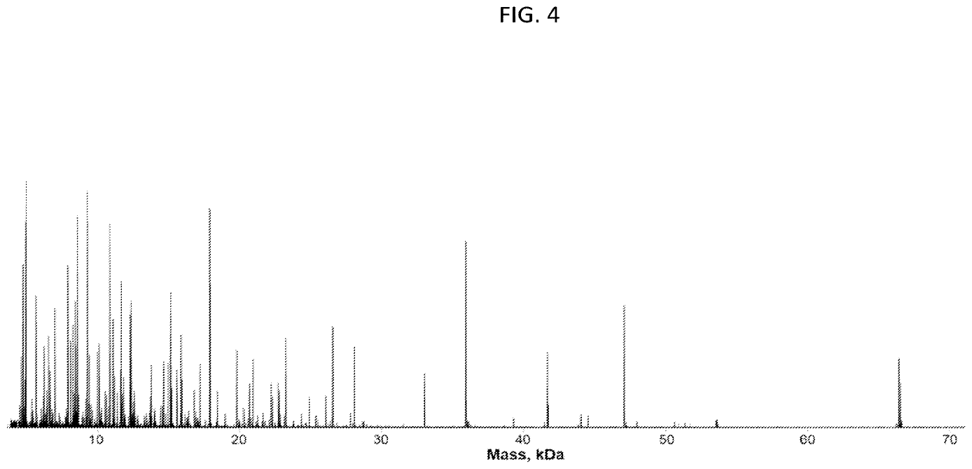

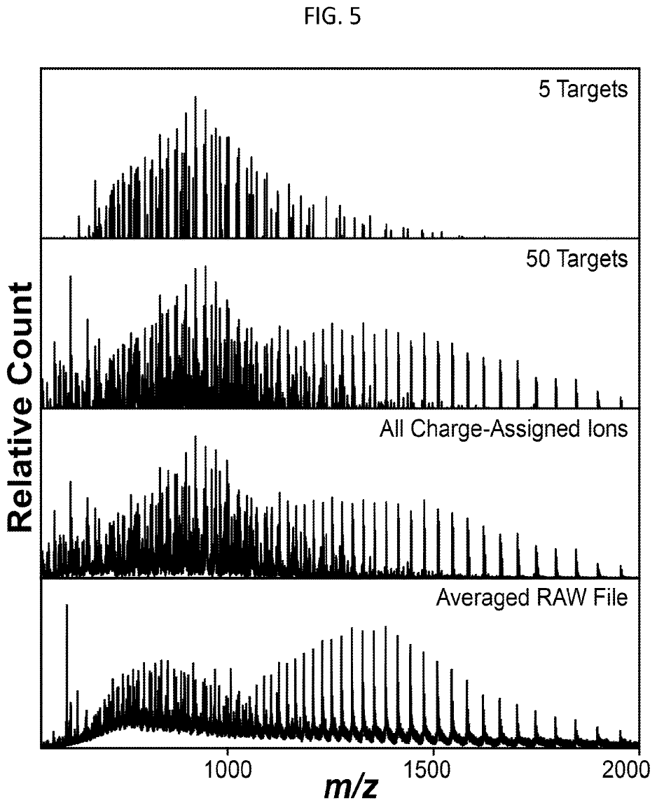

The disclosed technology may be applied to a thin section of sample tissue, e.g., a 10 μm thin section fresh-frozen human high-grade serous ovarian cancer (HGSOC) tissue (~95% tumor cellularity, FIG. 1a). As demonstrated by the Examples, 113 proteoform masses were obtained at >1% relative abundance ranging from 4-67 kDa from a single 40-minute survey line scan (FIG. 4, and Table 1), of which 25 were in the 17-50 kDa mass range (mass spectrum shown in FIG. 1b). The reconstructed survey scan shown in FIG. 5 illustrates the high spectral complexity in the m/z domain.

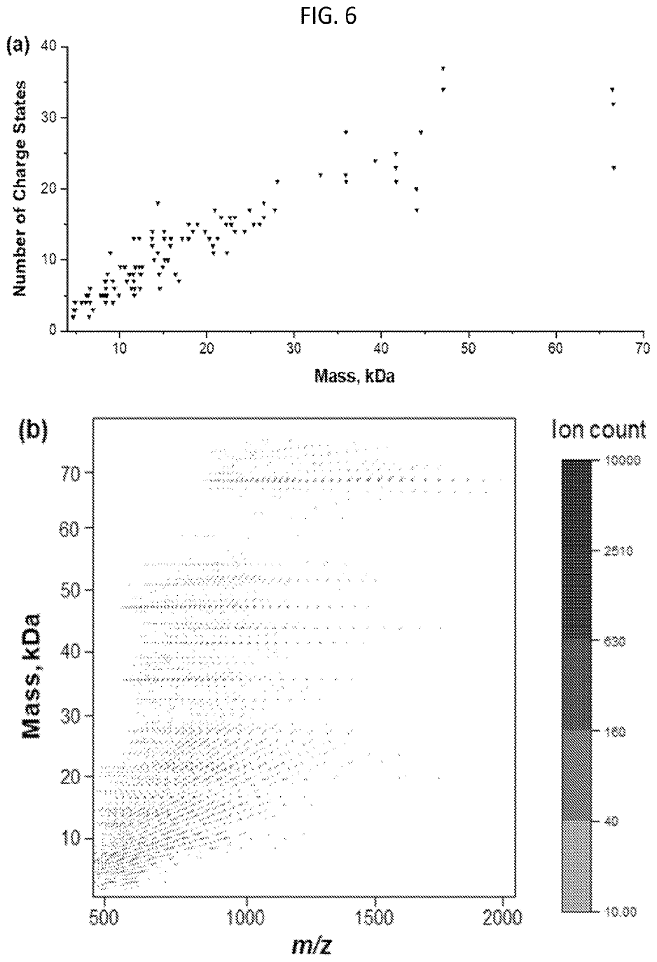

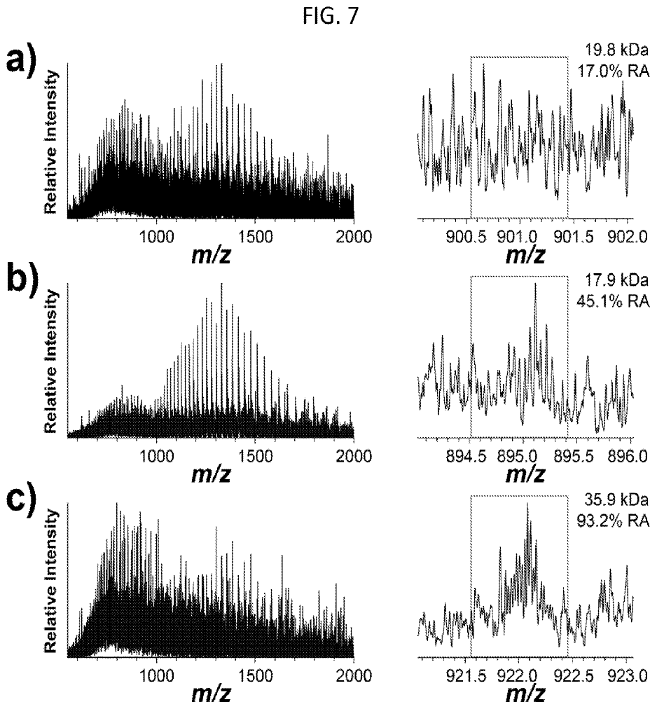

An algorithm may be engaged to direct data acquisition toward proteoform targets across both the m/z and spatial dimensions (where the target is at or near its highest abundance). As demonstrated by the Examples, the algorithm identified favorable m/z windows for 87 of the 113 proteoform masses (Table 2). Larger proteoforms are challenging targets, as their signals are diluted into many charge state and isotopic channels. Additionally, their signals often overlap with small proteoforms in the m/z domain (FIG. 6). However, the process was able to identify favorable m/z windows for all 25 targets (FIG. 1c and Table 2) at or near their top abundance in space (FIG. 1d and FIG. 7). Combined with the optimized spatial locations for these 25 targets, tandem MS data acquisition in I²MS mode and database searching identified 23 of the 25 targets (FIG. 1b), with the quality of proteoform information being at Level 2A or better using the five-level proteoform classification system. In addition, the disclosed technology allowed spatially-enhanced targeting of larger proteoforms. In FIG. 1e, the Examples show the mass spectrum and spatial distributions of two ~53.6 kDa proteoforms in a survey line scan from another HGSOC tissue specimen. The workflow generated MS² spectra containing sequence tags for identification using ≤100 MS² scans (FIG. 1e).

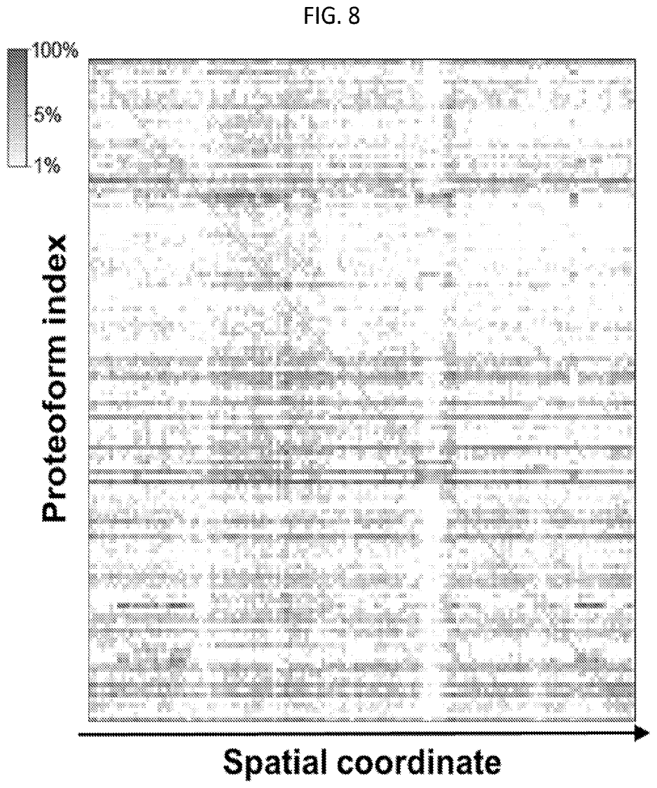

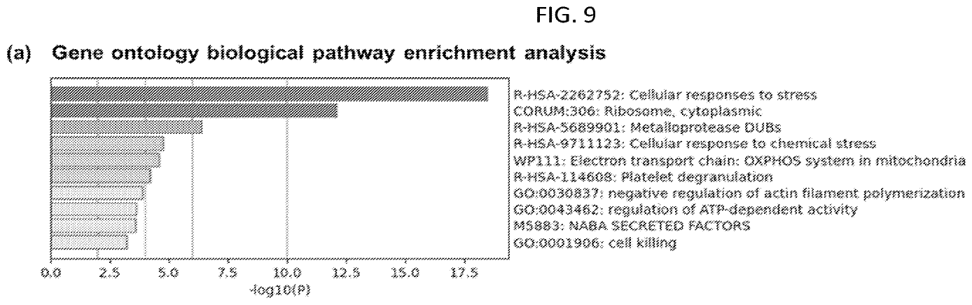

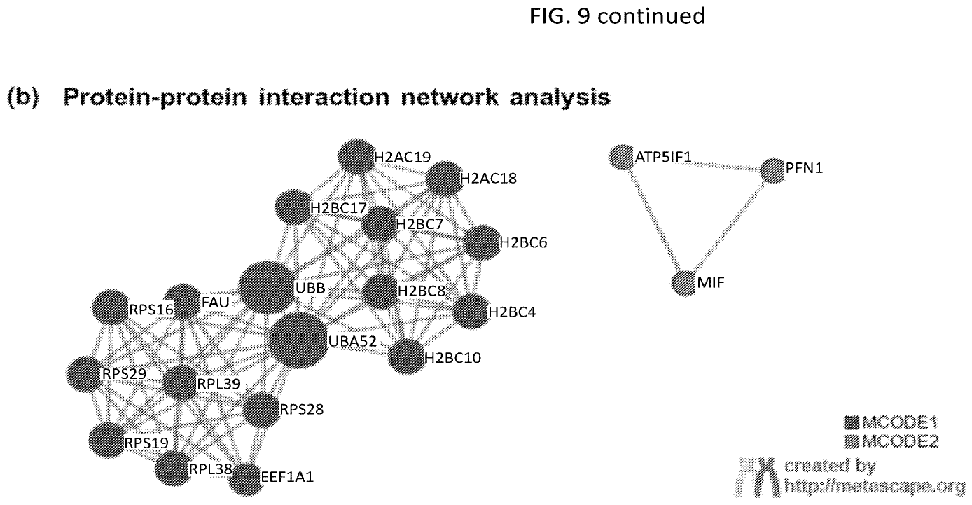

Also as demonstrated by the Examples, MS² in ensemble mode provides comparable information to MS² in I²MS mode for proteoforms with masses <17 kDa (FIG. 8). For a representative MS² line scan, matching candidates for 40 proteoforms above 1% relative abundance passing either a 1% false discovery rate or manual inspection were found. From a set of three technical replicates for proteoforms >17 kDa and three runs for proteoforms <17 kDa on the same HGSOC tumor tissue, the workflow identified a total of 73 proteoforms at the MS² level. A gene ontology analysis using the 73 proteoforms showed that <cellular responses to stress>, <ribosome, cytoplasmic>, and <metalloprotease deubiquitinases> were the highest enriched terms in HGSOC with p-values of 10⁻¹⁸, 10⁻¹², and 10⁻⁶, respectively (FIG. 1b and FIG. 7).

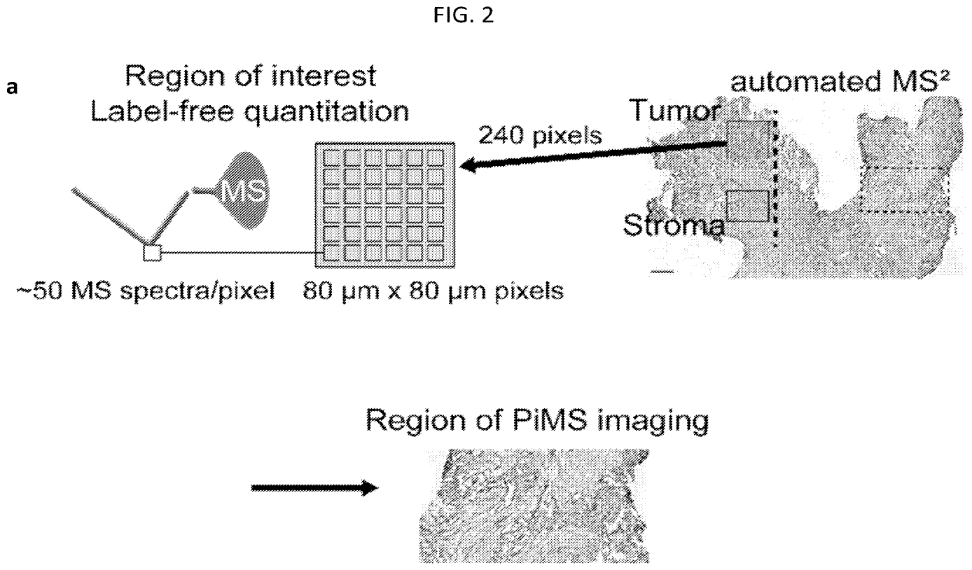

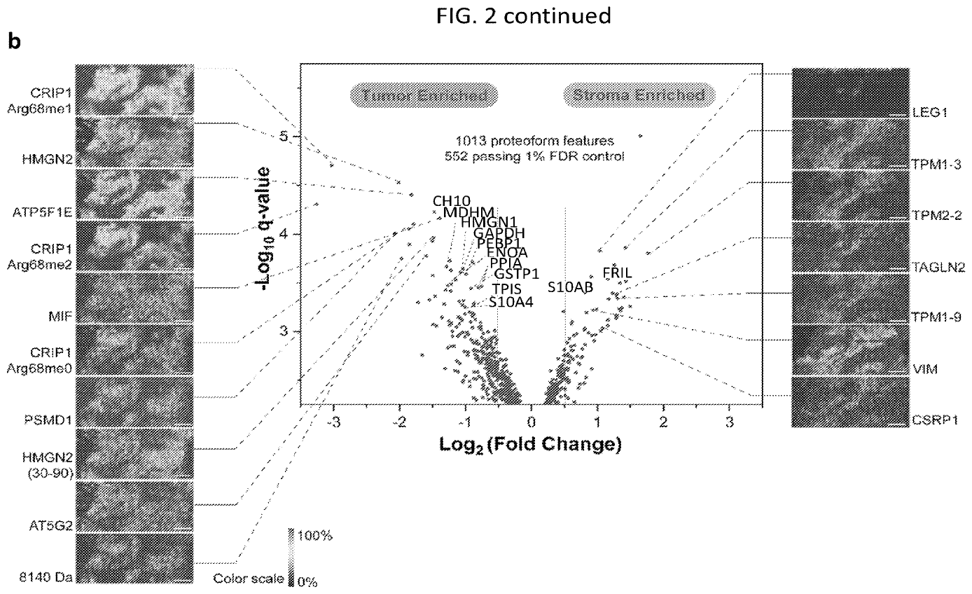

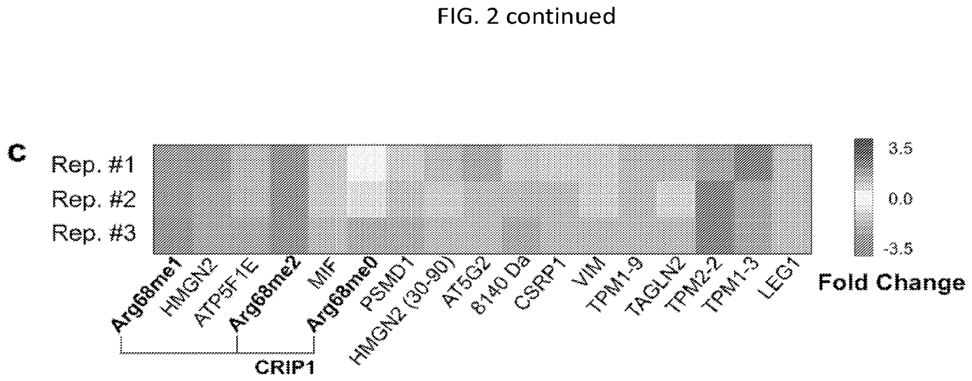

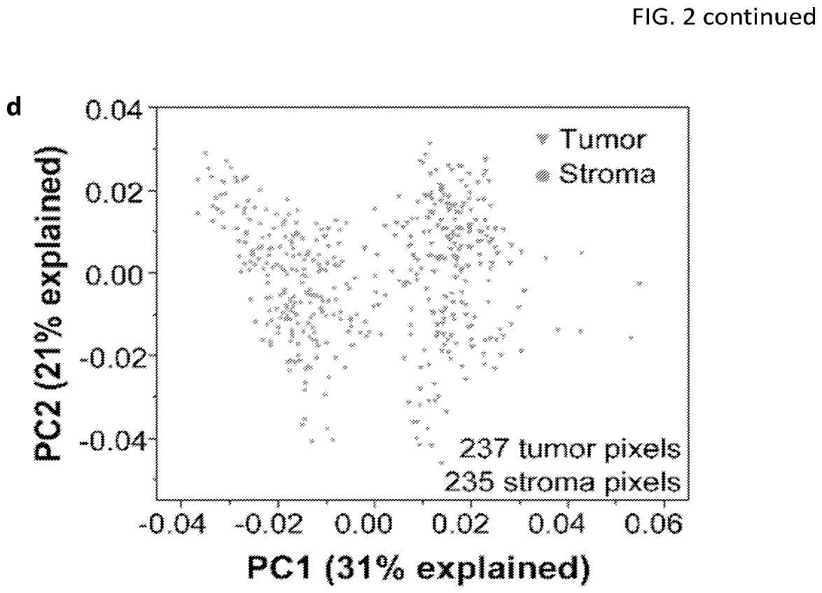

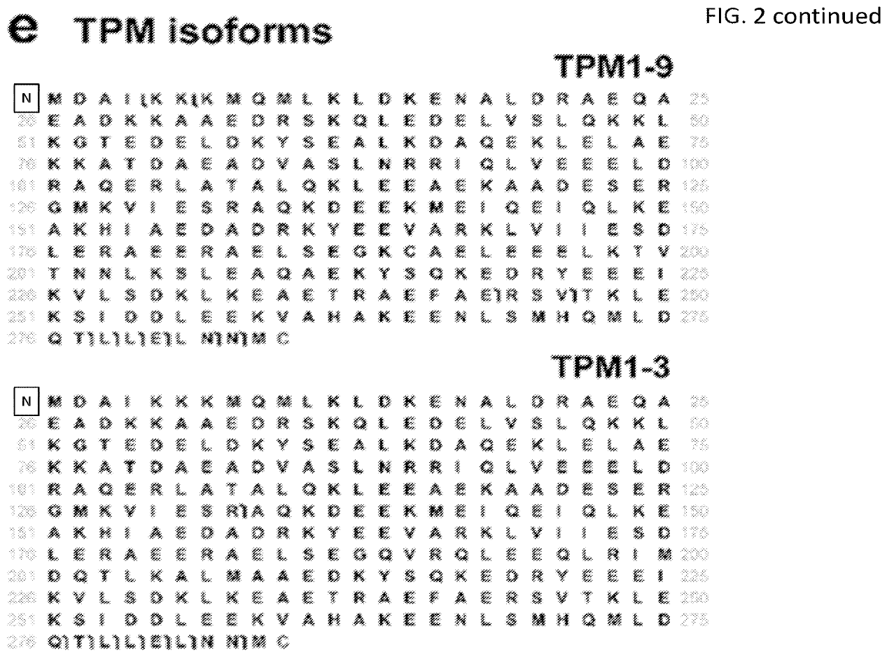

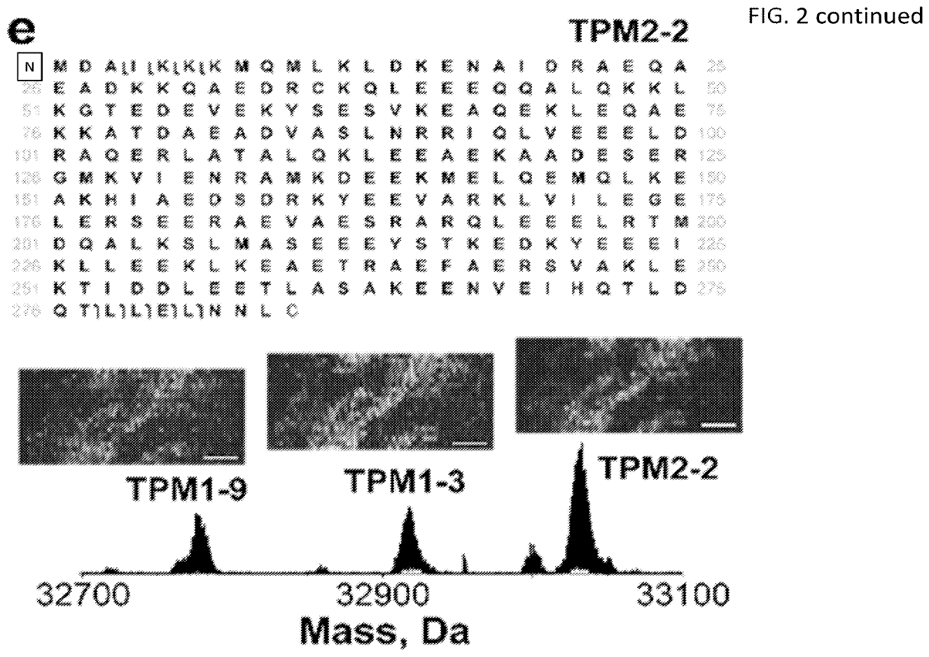

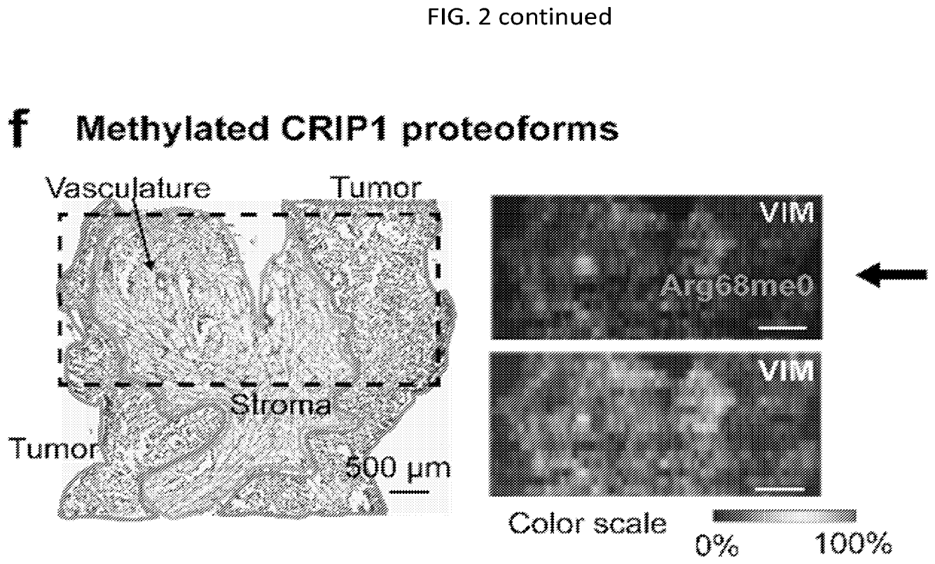

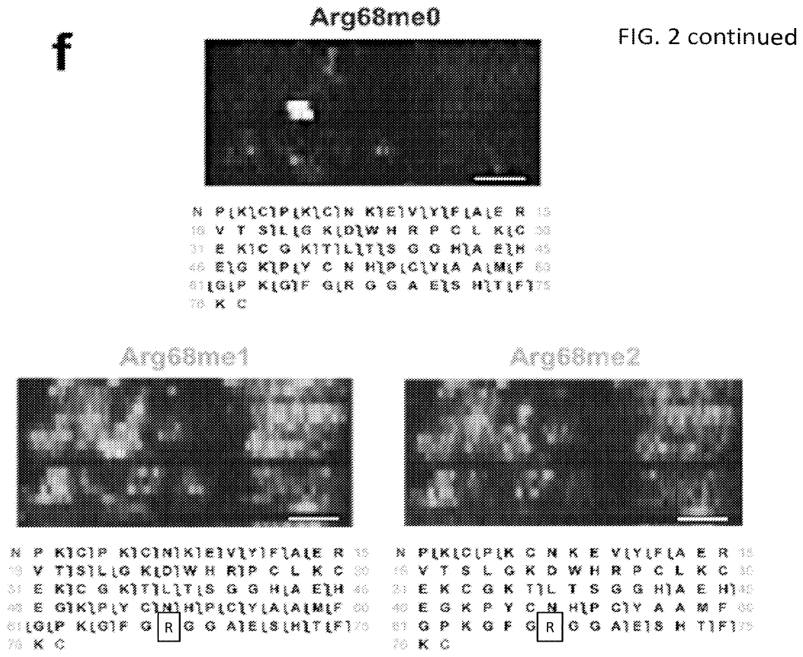

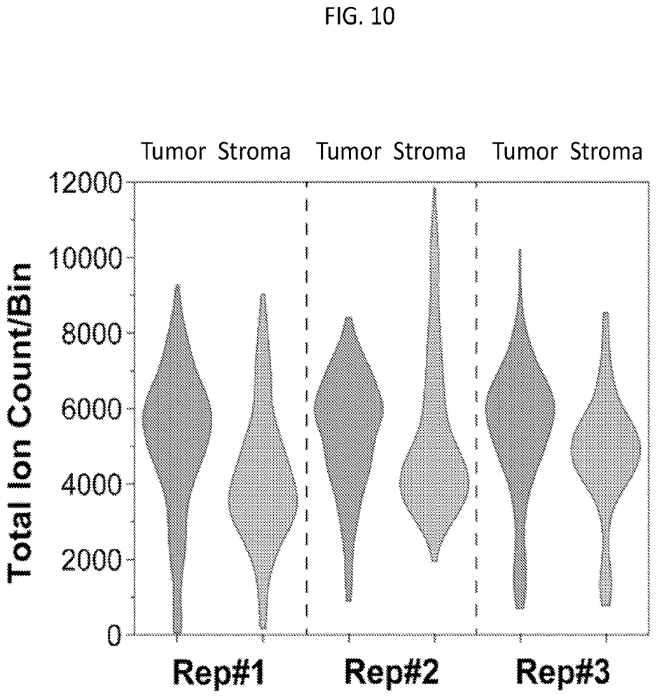

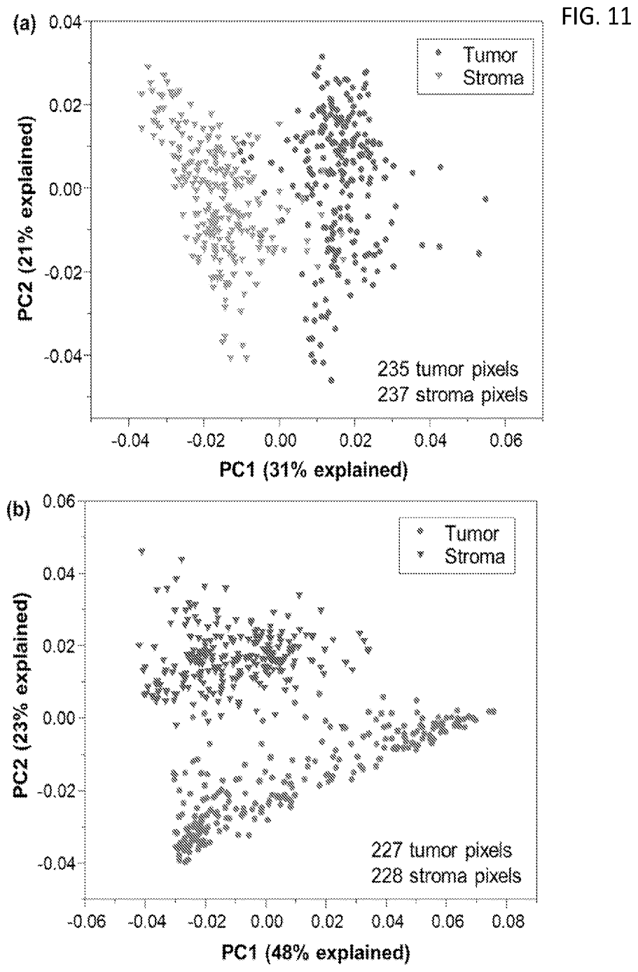

The disclosed technology may be utilized to discover proteoform signatures in sample tissue, such as histology-defined tumor versus stromal regions within HGSOC tissue from a single patient. A proteoform signature may include one or more of the following: intact molecular mass, spatial distribution, analysis of differential expression, or molecular identification of one or more proteoforms. A proteoform signature may include additional properties or characteristics of the one or more proteoforms in the sample tissue. In some instances, the proteoform signature comprises two or more, or all, of the following: intact molecular mass, spatial distribution, analysis of differential expression, and molecular identification of one or more proteoforms. Cancer-associated stroma co-mingles with cancer cells in tumor tissues in complex spatial arrangements and plays important roles in tumor cell invasion and metastasis. Proteoform signatures enriched in tumor versus stroma may be detected both using label-free quantitation (LFQ) or imaging (FIG. 2a). In the LFQ, pixels, e.g., 240 pixels, were selected from either tumor or stromal regions on the same section, and ~50 I²MS scans were obtained for each pixel (FIG. 2a). Total ion count distributions showed overall lower protein abundance in stroma (FIG. 10). A total of 1013 proteoform masses were detected, and 552 proteoforms showed statistically significant differential ion counts between tumor and stroma regions filtered by a conservative 1% false discovery rate. The tumor and stroma pixels were subjected to principal component analysis using the ion counts of the 552 proteoforms and were readily separated into distinct clusters (FIG. 2d). The statistical test and unsupervised classification were shown to be reproducible in two additional LFQ technical replicates obtained from adjacent tissue thin sections from the same HGSOC tumor tissue specimen (FIG. 11). The LFQ analysis identified 303 differentially abundant proteoforms between the tumor and stromal regions with |log 2 (fold change)|>0.5 (FIG. 2b). Images of 17 of these proteoforms (FIG. 2b) show distinct tumor or stromal localization with highly differential ion counts in LFQ. This result was highly consistent across the three technical replicates shown in FIG. 2c.

Proteoforms in this signature may be annotated using intact mass tag search against a database. The Examples demonstrate annotation of proteoforms from a database constructed from proteins found by the Clinical Proteomic Tumor Analysis Consortium (CPTAC) analyses of ovarian cancer. Using a stringent mass tolerance, e.g., 1.5 ppm, 114 proteoforms could be putatively identified using their intact mass from a set of ovarian cancer proteins.

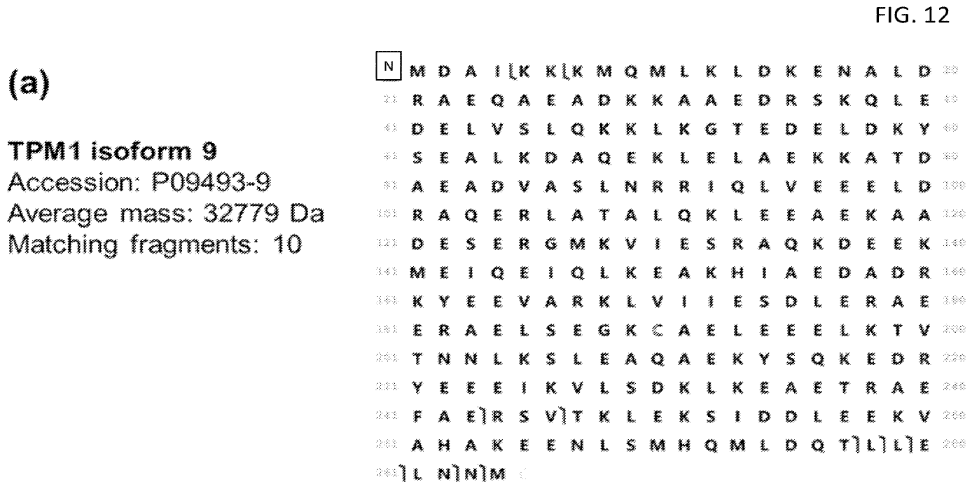

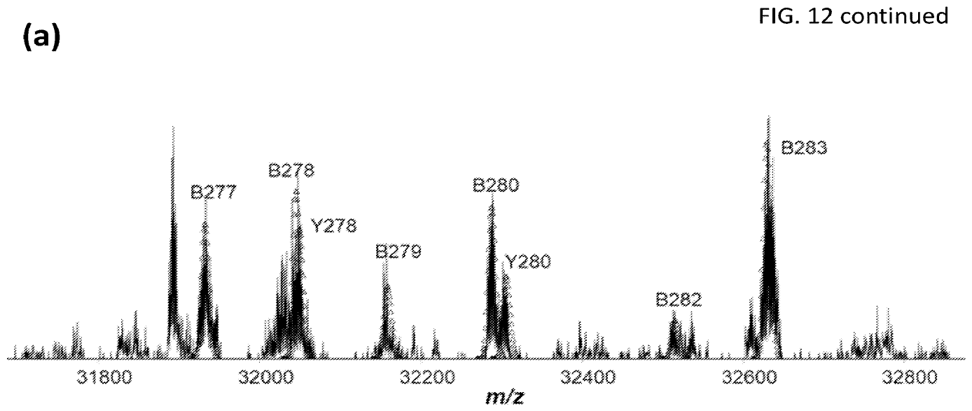

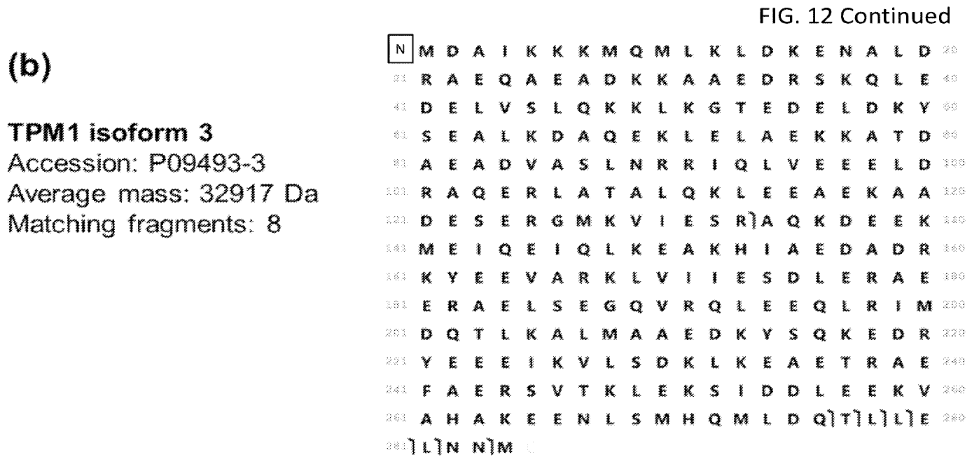

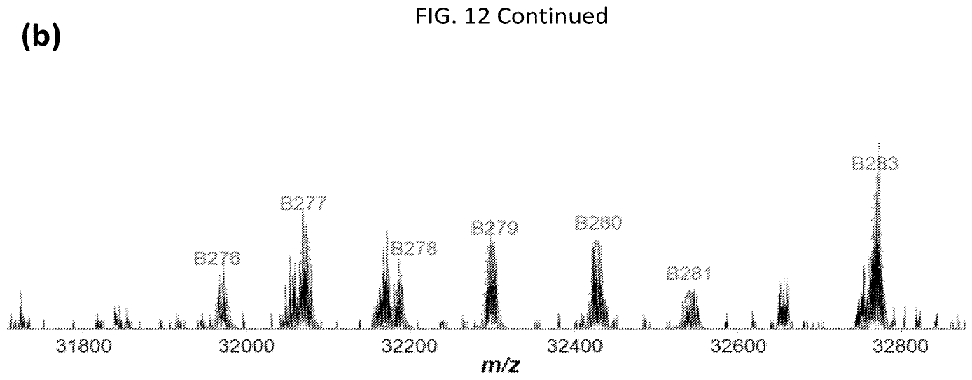

Furthermore, the technology was applied for direct top-down identification of signature proteoforms. Automated MS² method generation step pair with the LFQ output was used to selectively target proteoforms with the highest fold changes and highest confidences. In FIG. 2e, the Examples show the precise identification of tropomyosin alpha chain isoforms with >95% identical sequence known to exhibit fibroblast-specific expression (FIG. 12). The localization of these tropomyosin isoforms in imaging is consistent with fibroblasts being a major stromal cell type in the imaged region (FIG. 2b,e).

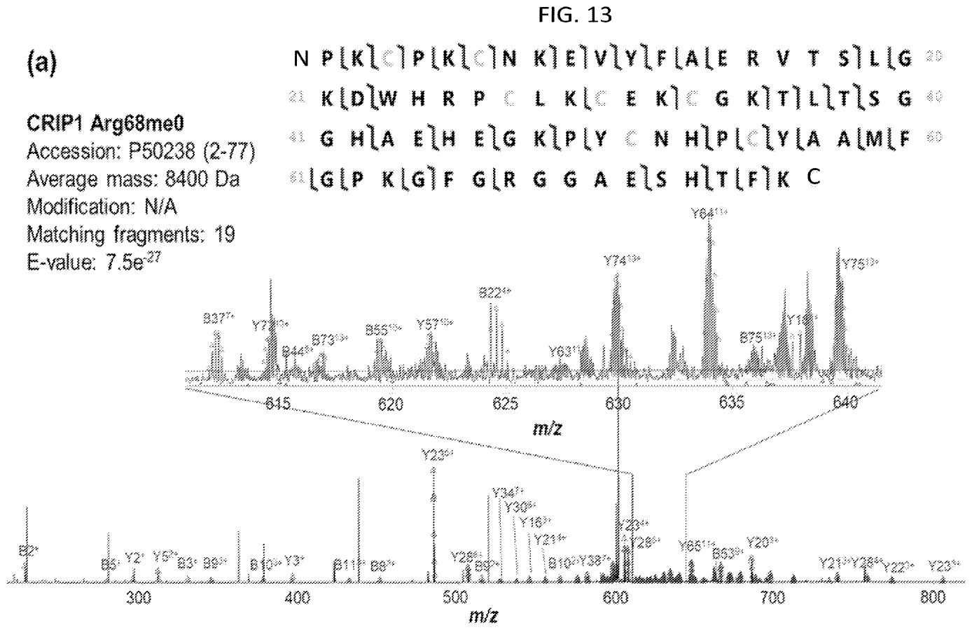

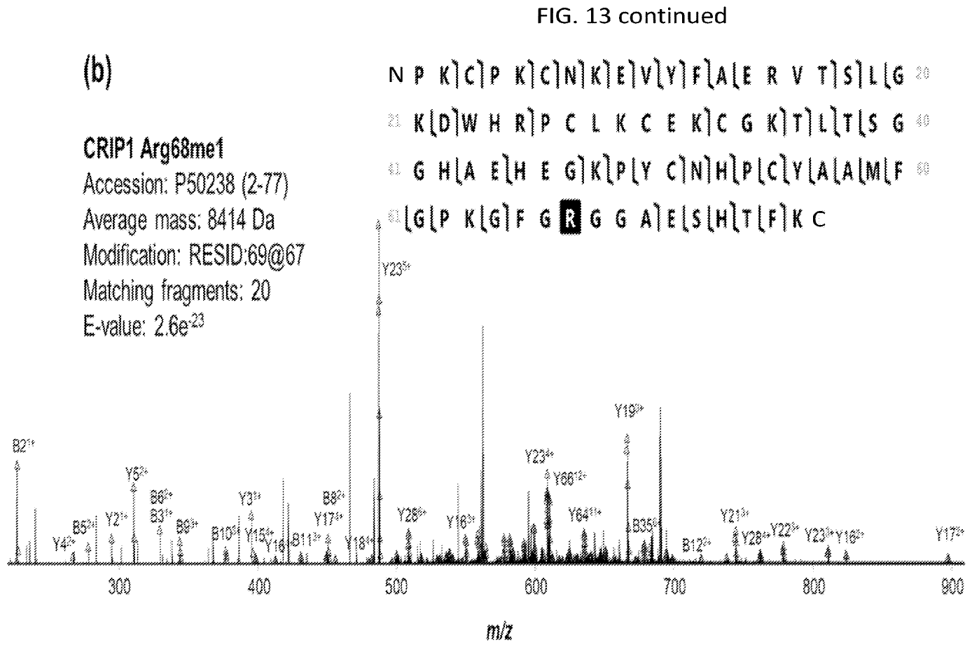

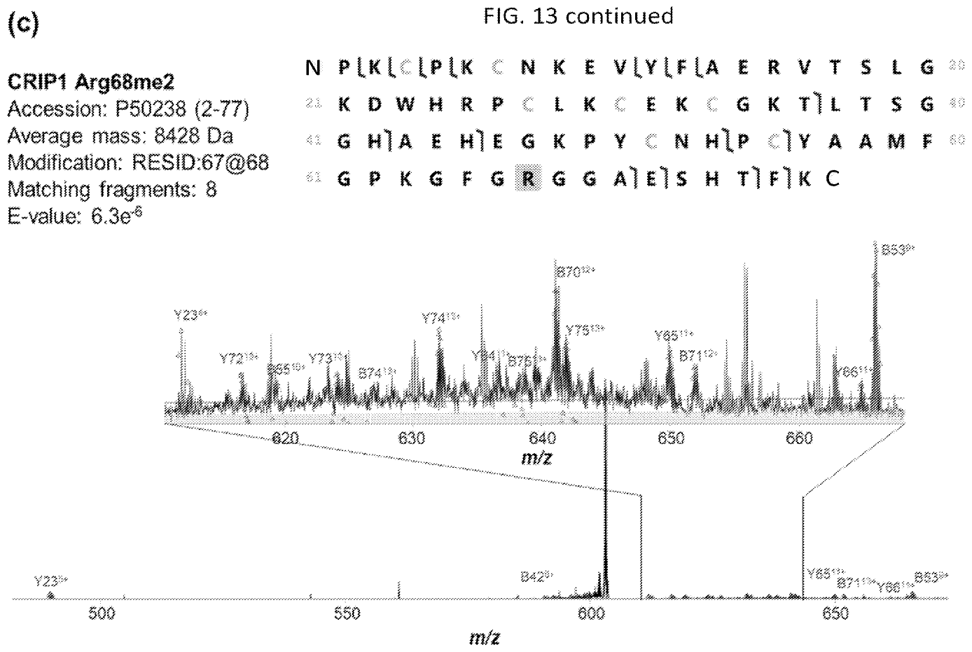

In the automated imaging experiment described above, post-translationally modified proteoforms may be identified. The Examples demonstrate that methylated proteoforms of the protein CRIP1 were detected at significantly higher levels in tumor regions and showed similar spatial distributions (FIG. 2b,c). The MS² data support the placement of the mono- and dimethylation on Arg68 (FIG. 2f and FIG. 13). Monomethylated CRIP1 is observed as the dominant CRIP1 proteoform in a majority of the locations on tissue. Notably, these three proteoforms showed differences in spatial distribution within the tumor region (FIG. 2f). Some vascularized locations in tumor showed higher relative levels of unmethylated CRIP1 whereas these regions excluded the Arg68me2 form of CRIP1 (FIG. 2f). Vascularized regions were asserted using microscopy images and co-localization of protein markers of cells comprising capillaries like vimentin (FIG. 2f). The higher level of dimethylation observed in large tumor regions may be attributed to PRMT overexpression or mutation-induced PRMT enzymatic activity. However, RNA-seq data from this patient does not show overexpression or mutation for any member of the PRMT gene family member.

In summary, the present technology is an integrated platform to drive advancements in proteoform-level spatial biology. The platform enables four-dimensional characterization of proteoform signatures: intact molecular mass, spatial distribution, quantitative analysis of differential expression, and molecular identification. In particular, the automated data acquisition engine enables proteoform identification up to ~54 kDa at a speed of <1 minute per proteoform directly off tissue. The Examples demonstrate ~300 differentially detected proteoforms in tumor versus stromal regions from ovarian cancer sections, where the patient can serve as their own control depending on the profiled regions in the experiment. This platform fills the gap between high-confidence proteoform discovery and spatial proteomics, opening up a new avenue for discoveries and precision diagnostics in clinical histology.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Tissue/Sample Preparation

Optimal cutting temperature (OCT)-embedded human high-grade serous ovarian cancer (HGSOC) tissues were cryo-sectioned (10 μm), thaw mounted onto glass microscope slides (Indium tin-oxide coated glass, Delta Technologies, CG-81 IN-S115, Fisher Scientific, Waltham, MA) and stored at −80 °C before mass spectrometry (MS) analysis. Detailed information of the tissue sections (H&E stained images, percentage of tumor, and tissue sites) used in this study are included in FIG. 14.

The HGSOC tissue sections were thawed at room temperature in a desiccator under slight vacuum, fixed and desalted via successive immersion in 70%/30%, 90%/10%, and 100%/0% (v/v) ethanol/water solutions for 20 s each, delipidated by 99.8% chloroform for 60 s, and dried under slight vacuum. The tissue sections were scanned using a PathScan Enabler (Fisher Scientific, Waltham, MA) prior to experiments.

Pierce Intact Protein Standard Mix was purchased from Fisher Scientific (Waltham, MA) and diluted in LC-MS grade 60%/39.5%/0.6% (v/v/v) water/acetonitrile/glacial acetic acid solvent to make a 5 ng/μL solution for direct infusion.

### PiMS Ion Source and Sampling Conditions

Proteoform imaging mass spectrometry (PiMS) utilizes nanospray desorption electrospray ionization (nano-DESI) MS for spatial profiling of thin tissue sections, which has been described in detail elsewhere. Briefly, the nano-DESI probe is comprised of a primary capillary to deliver extraction solvent to the tissue and a nanospray capillary for analyte transfer and ionization at MS. Localized analyte extraction from tissue is sampled by a dynamic liquid bridge formed between the junction of the two capillaries and the tissue section.

The PiMS probe used in this study was fabricated using fused silica capillaries (Molex, Thief River Falls, MN, OD/ID 150/75 μm and OD/ID 100/40 μm). To improve the spatial resolution of sampling, the primary capillary was flame-pulled to an OD of 20 μm on one side to make contact with the nanospray capillary (OD 100 μm, ID 40 μm). The width of the liquid junction formed by this probe was ~80 μm in all experiments estimated by measuring the width of the trace left by the probe on tissue using optical microscopy (data not shown). Region-of-interest (ROI) spatial profiling for label-free quantitation (LFQ), imaging, and fragmentation experiments were performed by moving the sample under the PiMS probe in lines, during which the analytes from different spatial locations were transferred to MS for detection. Line scans on tissue were performed at a rate of 3-5 μm/s. To improve the stability of the liquid bridge during spatial profiling, we first measured the surface tilting angle of the tissue section by defining a three-point plane on tissue prior to experiments. All samples were electrosprayed in positive ionization mode through the PiMS probe under denaturing conditions in a 60%/39.4%/0.6% (v/v/v) acetonitrile/water/glacial acetic acid solution at a flow rate of 300-400 nL/s.

### MS Settings and Data Acquisition

Data acquisition for fragmentation-based analysis for <17 kDa proteoforms, PiMS imaging, and LFQ was performed on an Orbitrap Exploris 480 mass spectrometer (Thermo Fisher Scientific, Bremen, Germany). Fragmentation using MS² in I²MS mode for >17 kDa proteoforms was conducted on a Q-Exactive Plus mass spectrometer (Thermo Fisher Scientific, Bremen, Germany). Data acquisition for Pierce Intact Protein Standard Mix was conducted either on a Q-Exactive Plus or a Q-Exactive HF mass spectrometer (Thermo Fisher Scientific, Bremen, Germany). The source conditions on the mass spectrometers were set as follows: ESI voltage: 3 kV; in-source CID: 0-15 eV; S-Lens/Funnel RF level: 70%; capillary temperature: 325 °C.

MS injection time is an important parameter in PiMS. We used 0.2-0.4 ms fixed injection time for PiMS imaging, MS² survey line scans, and LFQ experiments on the Exploris 480 mass spectrometer for the tissue sections presented in this study. 4 millisecond injection time was employed for MS² survey line scans on tissue on the Q-Exactive Plus mass spectrometer. Injection time for Pierce Intact Protein Standard Mix survey was fixed at 0.1 ms.

MS resolution was 120000 at m/z 200 for all MS¹ experiments conducted on the Orbitrap Exploris 480 mass spectrometer corresponding to 0.5 s Orbitrap detection period. The Orbitrap central electrode voltage was adjusted to 1 kV on the Q-Exactive Plus and Q-Exactive HF mass spectrometers to improve ion survival rate and resulting individual ion charge assignment. The MS resolution on Q-Exactive Plus was 140000 at m/z 200 corresponding to 2 s Orbitrap detection period for MS² survey line scans.

HCD pressure level was optimized on these instruments to reduce collision-induced ion decay within the Orbitrap analyzer without substantial losses in trapping efficiency in the C-trap. In particular, the HCD pressure setting was kept at 0.5 (arb.) for Q-Exactive Plus and Q-Exactive HF mass spectrometer, and 0.33 was used for Orbitrap Exploris 480 mass spectrometer (UHV pressure <5×10⁻¹¹ Torr). 0.5-1 V extended trapping was used on the Orbitrap Exploris 480 mass spectrometer to enhance the trapping efficiency. Additional relevant data acquisition parameters were adjusted as follows: mass range: 400-2500 m/z; AGC mode: fixed; enhanced Fourier transform: off; Emeter averaging: 0; microscans: 1.

### AutoPiMS Workflow and Automated MS² Algorithms

### Survey Scan and Target Selection Algorithm

Individual ion MS (12 MS) can resolve overlapping m/z components in the mass domain and detect species that would otherwise disappear into the proteinaceous signal baseline. However, this potential is realized as a result of data processing after acquisition and not in real time during acquisition. This combined with the fact that the measurement involves individual ions instead of clearly defined ensemble regime charge state distributions indicates that traditional data-dependent acquisition approaches are insufficient. Therefore, a precursor selection algorithm that capitalizes off of the resolution and sensitivity of the processed I²MS data is necessary.

First, a survey (i.e., intact) I²MS experiment is conducted on the sample (or, in the case of tissue imaging, a line scan parallel and adjacent to the line scan to undergo fragmentation). After processing the data via STORIBoard (Proteinaceous, Evanston, IL), the table of ions that constitute the resultant mass spectrum can be exported for further analysis.

The ion table is then processed by a fragmentation selection program (written in MATLAB in-house). A peak-picking algorithm is run through the I²MS mass spectrum to select proteoform precursors above a user-defined relative abundance threshold. These precursors can be filtered, e.g., by the user, to only target proteoforms of interest. Then, with the list of precursor masses, the algorithm assigns each ion to one of the target precursor masses, if possible. Specifically, if an ion's m/z measurement falls within the inclusion window for a given precursor mass (i.e., within +/−n Da of a mass identified by the peak-picking algorithm), the ion is assigned to that precursor mass. With these assignments, it is possible to reconstruct the m/z spectrum of each precursor, purified in silico.

After reconstructing the m/z spectra of all targets, the algorithm goes target by target, comparing the number of target ions in a given m/z window versus the number of other ions within that same window. Isolation windows for fragmentation for each species are selected on the basis of both target abundance and target window purity (i.e., the limited presence of co-isolating species). Then, during any subsequent and comparable experiments, the user can “blindly” (as in, not verifying with online, real-time MS¹ information) fragment at the noted m/z to characterize the target. Furthermore, the algorithm notes the abundance and mass identity of co-isolating species within each target m/z window, which allows the user to better account for fragmentation peaks that were not annotated as originating from the intended target.

### Spatial Pixel Allocation Algorithm

For samples that are homogenous across the entire experiment (e.g., direct infusion with a syringe pump), the target selection algorithm described above is sufficient, as it outputs a prioritized list of m/z regions for characterization. However, in the AutoPiMS workflow on dynamic samples such as tissue, the proteoform composition and concentration changes as the sampling probe moves to different locations in the tissue being sampled. Therefore, a complementary algorithm is required to prioritize the temporal (and, therefore, spatial) order in which target m/z windows are characterized.

The line region is divided into a number of equally-sized pixels depending on the number of targets. By default, one pixel is assigned one target. The algorithm first imports the reconstructed spatial profiles of the targets in the selected m/z windows identified in the previous section. The reconstructed spatial profiles detail ion counts corresponding to MS scans. MS scan indices are converted into spatial coordinates calculated from the constant, lateral motion of the PiMS probe and the MS data acquisition rate. Next, low-resolution spatial profiles are constructed to match the number of pixels of the line region. This is achieved by binning ion counts from scans located within the same pixel.

Next, the algorithm finds the pixel containing the highest ion counts for each target as its desired spatial assignment for MS². Some pixels may not be the highest ion count for any of the targets, which therefore are left unassigned. Pixels containing a unique target are immediately assigned to the target. Further optimizations are performed by the algorithm for pixels ideal for more than one target. In particular, the pixel is assigned to the target with the lowest absolute ion count among all the targets contained within this pixel. All the rest of the targets that do not find a pixel are returned to the pool for the next round to look for secondary pixel options. In this round, the algorithm finds the second highest ion count for the unassigned targets within the rest of the pixel indices and repeats the same decision making process as described above. The algorithm iterates the decision making process until all targets are each assigned a unique pixel. For the current experimental scale (i.e., containing up to ~100 targets), the algorithm reaches convergence within 10 iterations.

The algorithm is also able to accommodate MS² in I²MS mode for fewer targets, in which multiple pixels may be assigned to one target. This allows for targeting >17 kDa proteoforms using MS² in I²MS mode, which requires substantially more MS² scans to build ion statistics for fragment matching than proteoforms <17 kDa. In this version of the algorithm, the line region is divided into 150-300 μm pixels to represent the spatial variations of the proteoforms without sacrificing the targets with most desirable locations lumped in a large pixel. For current scale of the experiment, up to two pixels may be assigned to each target. Using the same logic, the algorithm finds the most favorable pixel for each target in the first step. The second step assigns one of the rest of the pixels to targets of the lowest total abundances following the same logic. In addition, the algorithm contains an add-in function to adjust the number of pixels for maximizing the prioritization of large proteoforms at relatively low abundances.

### MS² Method Setup

The algorithm generates a list of spatial pixels with assigned target m/z isolation windows corresponding to proteoform targets. The spatial locations of the pixels in the list are then converted into chronological events in a MS² acquisition sequence calculated by the probe scan rate (typically maintained at the same rate compared to the survey). The list is then imported into an instrument method in XCalibur (“Targeted MS²” on Orbitrap Exploris 480 or “DIA” on Q-Exactive Plus) for MS² data acquisition.

### Automated MS² Data Acquisition and Processing

MS² line scans in the automated MS² experiments were performed by shifting 20 μm longitudinally from the survey line scans (FIG. 1a). The closer spacing between the lines guarantees the similarity of the spatial protein abundances between the survey and the MS² sampling. MS² experiments can be performed in a spatially targeted manner without going through the entire line region (e.g., FIG. 1e). During the MS² line scan, targeted MS² experiments were performed using higher-energy collisional dissociation (HCD). For >17 kDa proteoform targets using automated fragmentation in I²MS mode on Q-Exactive Plus mass spectrometer, experiments were performed at MS resolution of 70000 at m/z 200 corresponding to an Orbitrap detection period of 1 s (HCD pressure setting=0.5).⁵ HCD collision energy and injection time were optimized to obtain fragments in the individual ion regime. Typical collision energy (CE) and injection time used in this study were 9-12 eV and 500-700 ms, respectively. MS² experiments for direct infusion of the Pierce Intact Protein Standard Mix targets were performed under similar MS conditions with an injection time of 1-60 ms. Additional parameters in MS² in I²MS mode were set as follows: Mass range: 200-2000 m/z; AGC mode: fixed; enhanced Fourier Transform enable: Off.

Ensemble MS² spectra were recorded for <17 kDa proteoform targets on an Orbitrap Exploris 480 mass spectrometer. MS² experiments were performed with a MS resolution of 30000 at m/z 200 corresponding to an MS acquisition rate of 14.5 spectra/s (HCD pressure setting=1). Typical HCD normalized collision energy (NCE) and maximum injection time used in this study were 25-50 eV and 40 ms, respectively. Additional parameters for MS² in ensemble mode were the same as that for I²MS mode except for enhanced Fourier Transform enable: On.

For >17 kDa proteoform targets, MS² data was first subjected to I²MS processing for fragment ion charge assignment and mass-domain spectrum construction as described previously. An automated pipeline was developed to find and sum all stori files from the dataset corresponding to the same target for I²MS processing to generate mass-domain spectra in the .mzML format. For the ensemble MS² data type for <17 kDa proteoform targets, an automated data import function was implemented in ProSight Native (Proteinaceous, Evanston, IL) to find and sum all the MS scans in the RAW file corresponding to the same target precursor mass for searching. The same function works for importing .mzML data for MS² data acquired in I²MS mode.

### Database Search Algorithm

ProSight Native (Proteinaceous, Evanston, IL) was used to search target proteoforms with the batch mode option in the Native Proteoform workflow. A UniProt.xml file containing all known Swiss-Prot accession numbers in the human protein database was input as the protein database (https://www.uniprot.org/proteomes/UP000005640). An annotated proteoform search was performed with a 100 Da precursor mass tolerance using a 10 ppm fragment mass tolerance. For MS² in I²MS mode datatype, a modified Thorough High Resolution Analysis of Spectra by Horn (THRASH) algorithm was applied to the spectra to determine fragment ion masses with a signal-to-noise cutoff of 3. All proteoform IDs were also manually validated. For MS² in ensemble mode datatype, we implemented a false discovery rate (FDR) control step for the targeted database search according to the Benjamini-Hochberg (B-H) procedure using the following equation:

$$B - H critical value = - {log}_{10} ( \frac{i * Q}{m} ) \qquad ( 1 )$$

Where i=ranking of the p-score, Q=user-defined FDR, m=total number of proteoforms in the database. We used Q=1%, m=1M to calculate the B-H critical value for p-score of each proteoform ID, and find the proteoform IDs with B-H critical value >p-score. These proteoform IDs were considered as passing the 1% FDR control. Additional proteoform IDs were manually validated using TDValidator (Proteinaceous, Evanston, IL). Subsequence search with a 10 ppm mass tolerance was supplemented to find candidate truncated proteoforms. All truncated proteoforms found in the subsequence search were manually validated using TDValidator. A minimum of 3 matching fragment ions were required for a candidate proteoform to be returned as a hit. Proteoform IDs that did not pass the 1% FDR control and manual validation were listed as “Non-FDR-controlled candidate proteoforms.” TD Validator options for fragment annotation are listed as followings: Validator type: “Shape”/“Simple” (MS² in I²MS/ensemble mode); Max PPM Tolerance: 5; Sub PPM Tolerance: 3; Minimum Score: 0.25/0.5 (MS² in I²MS/ensemble mode).

MS¹-Level I²MS Methodology

### PiMS Data Acquisition and Processing

This section describes the methodology of MS¹-level spatial profiling that applies to imaging, LFQ, and the survey line scans that direct fragmentation. The varying parameters of these experiments are described in detail in the corresponding sections. MS¹-level data acquisition was performed in the I²MS mode described in detail elsewhere. In short, individual ions on tissue were collected by the PiMS probe as a function of locations; time-domain data files were acquired and recorded as Selective Temporal Overview of Resonant Ions (STORI) files. The STORI slope of an individual ion was compared to a charge calibration curve of the instrument, and an integer charge number (z) was assigned to the ion by statistically evaluation using an iterative voting algorithm. The neutral mass of the ion is calculated by:

$$Mass = ( \frac{m}{z} \times z ) - ( z \times {M}_{proton} ) \qquad ( 2 )$$

Neutral centroid masses of the individual ions were converted to profiles using a Kernel density estimation approach, and ions from the entire imaged area were used to construct a mass-domain spectrum. Proteoform features were identified in the spectrum using an iterative peak picking algorithm using a user-defined threshold and confirmed by a modified THRASH algorithm.

### PiMS Imaging

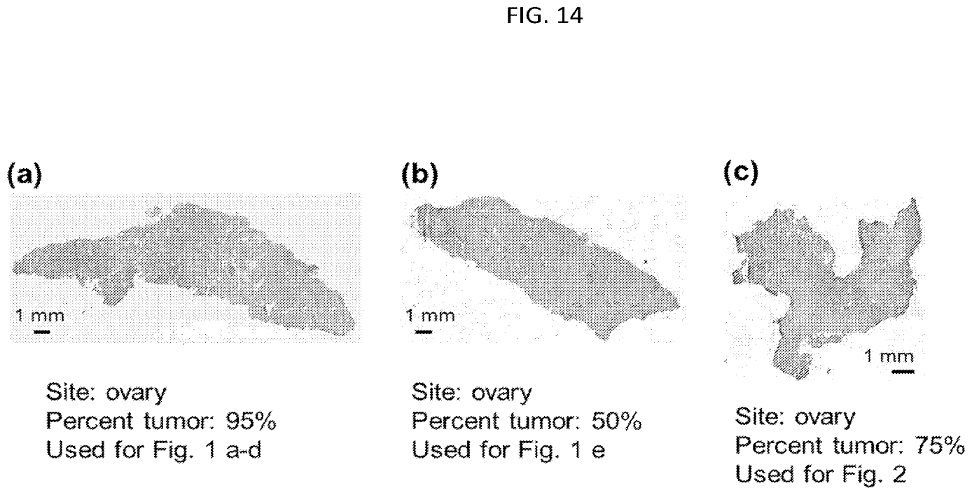

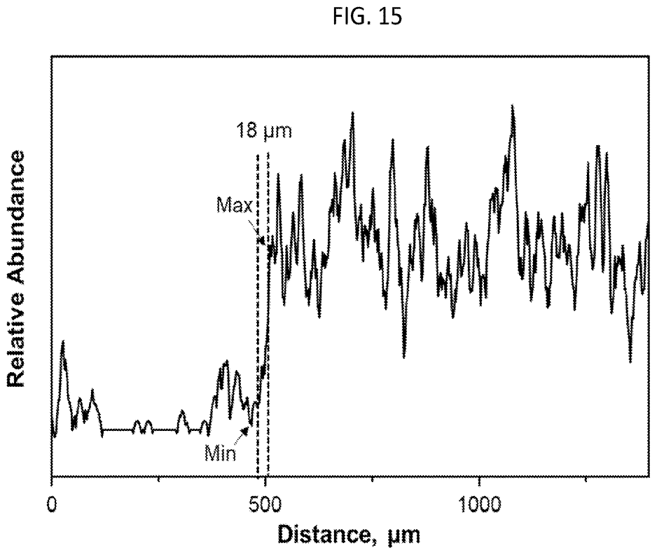

The PiMS imaging experiment shown in FIG. 2 was performed at a probe line scan rate of 5 μm/s and a data acquisition rate of 2 spectra/s with a strip step of 80 μm between the adjacent lines. The dimension of the two imaged regions in FIG. 2 were 3.4 mm×1.6 mm and were both composed of 20 line scans. PiMS proteoform images were generated using ions within the isotopic distributions of the proteoforms using a MATLAB script developed in-house. Major isotopic masses (5-11 isotopes depending on the proteoform mass) of a proteoform was first identified from the mass spectrum. A+10 ppm mass tolerance was used to find individual ions within the major isotopic masses composing the proteoform. The spatial locations of these ions were used to generate a two-dimensional heatmap as the proteoform image. The spatial resolution of the imaging experiments were ~20 μm (FIG. 15).

### Label-Free Quantitation (LFQ)

In the LFQ experiments, we analyzed 240 pixels (80 μm×80 μm) each in selected tumor and stroma regions of interest from the biopsy annotated by a histologist. In contrast to conventional LFQ sample preparation, each pixel was considered as an LFQ “sample”. The size of the pixel was chosen based on the footprint size of the probe. The experiments were performed at a probe scan rate of 4 μm/s and a spectral acquisition rate of 2 spectra/s with a strip step of 100 μm between adjacent lines. 100 μm strip step was employed instead of 80 μm to avoid repeated sampling of the same regions, which is detrimental to quantitation. Groups of 45 adjacent MS scans were binned to construct the proteoform profile of a sample, from which the proteoform ion counts were extracted. Charge assignment of the ions was conducted by co-evaluating all the 240 tumor and stroma samples together to retain the unsupervised nature of the analysis. Proteoform feature extraction from the sum of 480 samples >0.1% relative abundance results in similar number of fractures compared to that obtained using THRASH algorithm. Due to current limitations in relative quantitation, THRASH was used only for validation but not involved in the LFQ process. Using only major isotopes for quantitation guarantees the low-interference from adjacent overlapping proteoforms in the spectrum.

Before statistical analysis, a quality control step was performed to eliminate pixels with ion counts below a certain threshold (considered as empty samples due to probe lost contact or localized sample contamination). In this study, pixels with <1000 total ions were tossed out. From this step, 237 tumor and 235 stroma pixels (out of 240) passed the control and were used for further analysis. In another two technical replicates, 235/237 and 227/228 tumor/stroma pixels passed for further analysis.

Next, for each proteoform feature, a T-test was performed between the tumor and stroma pixels, from which a p-score was obtained. The p-scores are converted to FDR-controlled q-values using the Benjamini-Hochberg procedure. In particular, we first ranked the proteoforms according to −log₁₀ (p-score) in a descending order, and calculated the B-H critical scores (q-values) using Eq. (1), where i=ranking of the p-score, Q=user-defined FDR, and m=total number of pixels in tumor/stroma. All proteoforms with −log₁₀(p-score) higher than the critical value were considered statistically significant. In this study, we calculated the critical value using 1% FDR, which resulted in 552 out of the 1013 proteoforms passing the test. In another two technical replicates, we found 616 out of 939 and 597 out of 954 proteoforms to be statistically significant under same conditions. Fold change of each proteoform was calculated using the mean ion count of that proteoform across all stroma pixels divided by the mean ion count across all tumor pixels. A volcano plot in FIG. 2b was constructed by plotting-log 10 (q-values) of the statistically significant differential 552 proteoforms against log 2 (fold change).

In the next step, unsupervised linear dimension reduction using principal component analysis (PCA) was performed using the statistically significant proteoforms. In this workflow, PCA was employed to validate the clustering of tumor and stromal pixels. The workflow may be further adapted into studies with more than two biological sample. The proteoform ion counts were normalized to total ion count from a sample and subjected to PCA using a MATLAB script built in-house. The first two columns of the score table were extracted to generate a scatter plot displaying the separation of tumor and stroma clusters using the first two principal components. In addition, the MATLAB script built for LFQ is bridged to automated MS² acquisition in AutoPiMS. In particular, the target list in automated MS² can be constructed from the most significant features in the volcano plot obtained from LFQ.

### Intact Mass Tag (IMT) Search & Gene Ontology (GO) Analysis

Mass-domain spectra from the LFQ experiment were converted to mzML format and processed using a custom version of TDValidator (Proteinaceous, Evanston, IL) implemented with an MS¹ IMT search function. The spectrum was systematically calibrated according to the accurate masses of six MS/MS identified proteoforms in the 4-50 kDa mass range. The spectrum was searched against an ovarian cancer database, which was generated by the National Cancer Institute Clinical Proteomic Tumor Analysis Consortium (CPTAC) containing 9239 proteins (CPTAC, PDC study ID: PDC000113 & PDC000114). Methionine on/off, water loss, monoacetylation, monophosphorylation and their one-to-one combinations were considered as possible proteoform modifications in the database. IMT search was performed with a ±1.5 ppm mass tolerance.

GO analysis was performed using Metascape (https://metascape.org/). Specifically, a list of Entrez Gene IDs was retrieved for the 73 MS² identified proteoforms from Uniprot and submitted to Metascape for GO analysis. The result contains the top-level enriched GO biological pathways and Protein-protein interaction (PPI) networks.

### Tables

TABLE 1
Proteoform masses at >1% relative abundance ranging
from 4-67 kDa from a single 40-minute survey line scan
obtained from a 10 μm thin section of fresh-frozen
human high-grade serous ovarian cancer (HGSOC) tissue.

| | Ion | Relative |
|---|---|---|
| Mass | count | Abundance |
| 66438.71 | 21059 | 100.0% |
| 35921.01 | 19631 | 93.2% |
| 47079.9 | 18269 | 86.8% |
| 66536.72 | 11206 | 53.2% |
| 17880.19 | 9488 | 45.1% |
| 41661.33 | 7703 | 36.6% |
| 26538.16 | 6439 | 30.6% |
| 17923.02 | 6174 | 29.3% |
| 35903.88 | 5633 | 26.7% |
| 28078.87 | 5519 | 26.2% |
| 47062.01 | 5136 | 24.4% |
| 10843.05 | 4965 | 23.6% |
| 33000.84 | 4793 | 22.8% |
| 15126.05 | 4602 | 21.9% |
| 23224.43 | 4375 | 20.8% |
| 12344.46 | 4027 | 19.1% |
| 20925.84 | 3760 | 17.9% |
| 15866.55 | 3598 | 17.1% |
| 19800.85 | 3587 | 17.0% |
| 41647.22 | 3521 | 16.7% |
| 11651.07 | 3477 | 16.5% |
| 23210.32 | 3320 | 15.8% |
| 35954.02 | 3259 | 15.5% |
| 8564.862 | 3220 | 15.3% |
| 11637.97 | 3020 | 14.3% |
| 9261.269 | 2966 | 14.1% |
| 12275.42 | 2588 | 12.3% |
| 41717.26 | 2574 | 12.2% |
| 17209.23 | 2453 | 11.6% |
| 11071.57 | 2406 | 11.4% |
| 10091.46 | 2337 | 11.1% |
| 22692.8 | 2282 | 10.8% |
| 14964.8 | 2196 | 10.4% |
| 20688.49 | 2191 | 10.4% |
| 26062.47 | 1993 | 9.5% |
| 13761.21 | 1962 | 9.3% |
| 4963.651 | 1919 | 9.1% |
| 15561.18 | 1901 | 9.0% |
| 24901.45 | 1866 | 8.9% |
| 22204.51 | 1815 | 8.6% |
| 22772.67 | 1779 | 8.4% |
| 14626.43 | 1705 | 8.1% |
| 22302.52 | 1553 | 7.4% |
| 4935.684 | 1525 | 7.2% |
| 44524.81 | 1514 | 7.2% |
| 18413.83 | 1454 | 6.9% |
| 7883.32 | 1444 | 6.9% |
| 66601.72 | 1442 | 6.8% |
| 8415.2 | 1368 | 6.5% |
| 11603.96 | 1294 | 6.1% |
| 44018.89 | 1260 | 6.0% |
| 16789.97 | 1255 | 6.0% |
| 15929.79 | 1238 | 5.9% |
| 27787.11 | 1194 | 5.7% |
| 9954.148 | 1126 | 5.3% |
| 44030.73 | 1086 | 5.2% |
| 11820.14 | 1079 | 5.1% |
| 17864.06 | 1037 | 4.9% |
| 15195.09 | 1002 | 4.8% |
| 8451.733 | 971 | 4.6% |
| 26520.02 | 960 | 4.6% |
| 8245.633 | 953 | 4.5% |
| 13712.33 | 943 | 4.5% |
| 12580.79 | 942 | 4.5% |
| 11181.68 | 940 | 4.5% |
| 39287.9 | 939 | 4.5% |
| 17905.13 | 924 | 4.4% |
| 15109.17 | 919 | 4.4% |
| 6977.033 | 909 | 4.3% |
| 9408.411 | 801 | 3.8% |
| 44043.83 | 790 | 3.8% |
| 4735.379 | 778 | 3.7% |
| 11728.93 | 777 | 3.7% |
| 15142.18 | 761 | 3.6% |
| 24334.05 | 753 | 3.6% |
| 5647.208 | 729 | 3.5% |
| 21640.38 | 715 | 3.4% |
| 4747.472 | 694 | 3.3% |
| 6545.432 | 680 | 3.2% |
| 8087.908 | 669 | 3.2% |
| 6223.432 | 664 | 3.2% |
| 11382.24 | 654 | 3.1% |
| 22852.8 | 650 | 3.1% |
| 20288.89 | 599 | 2.8% |
| 10613.52 | 585 | 2.8% |
| 11712.05 | 566 | 2.7% |
| 15849.42 | 564 | 2.7% |
| 12367.39 | 563 | 2.7% |
| 12259.55 | 530 | 2.5% |
| 9246.151 | 517 | 2.5% |
| 8400.082 | 491 | 2.3% |
| 21261.44 | 479 | 2.3% |
| 14550.59 | 478 | 2.3% |
| 14424.61 | 477 | 2.3% |
| 25365.56 | 446 | 2.1% |
| 16405.49 | 444 | 2.1% |
| 15833.04 | 443 | 2.1% |
| 13774.81 | 438 | 2.1% |
| 12501.68 | 413 | 2.0% |
| 18953.27 | 399 | 1.9% |
| 20775.17 | 393 | 1.9% |
| 8578.719 | 381 | 1.8% |
| 8429.057 | 362 | 1.7% |
| 6647.978 | 346 | 1.6% |
| 14413.02 | 342 | 1.6% |
| 13980.66 | 337 | 1.6% |
| 6157.42 | 312 | 1.5% |
| 6629.586 | 289 | 1.4% |
| 8326.511 | 285 | 1.4% |
| 8649.771 | 267 | 1.3% |
| 6432.556 | 256 | 1.2% |
| 4619.479 | 256 | 1.2% |
| 8959.677 | 242 | 1.1% |

TABLE 2
AutoPiMS workflow algorithm-identified favorable m/z windows
for 87 of the 113 proteoform masses shown Table 1.

| Mass | Average | | Selected | Component | Coisolated | Component |
|---|---|---|---|---|---|---|
| List ID | Mass | m/z | Charge State | Ion Count | Ion Count | Percentage |
| 1 | 66438.7083 | 1477.424 | 45 | 932 | 0 | 100% |
| 2 | 35921.0114 | 1382.585 | 26 | 122 | 0 | 100% |
| 4 | 66536.7193 | 1513.206 | 44 | 612 | 0 | 100% |
| 5 | 17880.1881 | 1052.784 | 17 | 764 | 0 | 100% |
| 7 | 26538.1607 | 1327.916 | 20 | 168 | 0 | 100% |
| 10 | 28078.8732 | 1170.961 | 24 | 165 | 0 | 100% |
| 13 | 33000.8385 | 1179.609 | 28 | 109 | 0 | 100% |
| 15 | 23224.4321 | 1452.535 | 16 | 46 | 0 | 100% |
| 16 | 12344.4573 | 1235.454 | 10 | 83 | 0 | 100% |
| 22 | 23210.3225 | 1366.321 | 17 | 46 | 0 | 100% |
| 23 | 35954.0177 | 1240.802 | 29 | 30 | 0 | 100% |
| 28 | 41717.265 | 1098.831 | 38 | 221 | 0 | 100% |
| 29 | 17209.2286 | 1230.238 | 14 | 21 | 0 | 100% |
| 32 | 22692.8044 | 1135.648 | 20 | 107 | 0 | 100% |
| 34 | 20688.4924 | 1150.369 | 18 | 164 | 0 | 100% |
| 35 | 26062.4673 | 1242.078 | 21 | 147 | 0 | 100% |
| 39 | 24901.4529 | 1132.892 | 22 | 89 | 0 | 100% |
| 41 | 22772.6745 | 1266.156 | 18 | 34 | 0 | 100% |
| 42 | 14626.4251 | 1045.753 | 14 | 311 | 0 | 100% |
| 43 | 22302.5242 | 1174.825 | 19 | 107 | 0 | 100% |
| 45 | 44524.8135 | 1114.128 | 40 | 41 | 0 | 100% |
| 48 | 66601.724 | 1257.644 | 53 | 135 | 0 | 100% |
| 52 | 16789.9734 | 1292.544 | 13 | 213 | 0 | 100% |
| 53 | 15929.7947 | 569.9291 | 28 | 82 | 0 | 100% |
| 54 | 27787.1078 | 1390.363 | 20 | 24 | 0 | 100% |
| 61 | 26520.0198 | 1206.463 | 22 | 9 | 0 | 100% |
| 63 | 13712.3278 | 980.4598 | 14 | 48 | 0 | 100% |
| 64 | 12580.7923 | 1144.716 | 11 | 15 | 0 | 100% |
| 66 | 39287.9028 | 1268.36 | 31 | 15 | 0 | 100% |
| 67 | 17905.1317 | 1194.683 | 15 | 9 | 0 | 100% |
| 73 | 11728.9282 | 1067.274 | 11 | 114 | 0 | 100% |
| 75 | 24334.0474 | 1432.422 | 17 | 8 | 0 | 100% |
| 77 | 21640.3832 | 1443.7 | 15 | 6 | 0 | 100% |
| 80 | 8087.9084 | 675.0002 | 12 | 142 | 0 | 100% |
| 84 | 20288.8898 | 752.4482 | 27 | 57 | 0 | 100% |
| 92 | 21261.441 | 1064.08 | 20 | 78 | 0 | 100% |
| 95 | 25365.5563 | 1103.858 | 23 | 27 | 0 | 100% |
| 97 | 15833.0435 | 609.971 | 26 | 6 | 0 | 100% |
| 98 | 13774.8129 | 919.3287 | 15 | 23 | 0 | 100% |
| 99 | 12501.678 | 1137.524 | 11 | 28 | 0 | 100% |
| 100 | 18953.2697 | 558.4569 | 34 | 11 | 0 | 100% |
| 101 | 20775.1654 | 1299.456 | 16 | 17 | 0 | 100% |
| 105 | 14413.0181 | 848.8324 | 17 | 28 | 0 | 100% |
| 106 | 13980.6612 | 874.7991 | 16 | 8 | 0 | 100% |
| 111 | 6432.5559 | 644.2634 | 10 | 75 | 0 | 100% |
| 26 | 9261.2687 | 579.8371 | 16 | 895 | 2 | 100% |
| 18 | 15866.5537 | 934.3345 | 17 | 450 | 2 | 100% |
| 30 | 11071.5743 | 652.2769 | 17 | 261 | 2 | 99% |
| 37 | 4963.6509 | 621.4642 | 8 | 840 | 8 | 99% |
| 38 | 15561.1828 | 865.518 | 18 | 344 | 4 | 99% |
| 57 | 11820.1364 | 696.31 | 17 | 176 | 2 | 99% |
| 49 | 8415.1996 | 562.0211 | 15 | 384 | 5 | 99% |
| 47 | 7883.3199 | 607.4171 | 13 | 524 | 10 | 98% |
| 62 | 8245.633 | 589.9816 | 14 | 412 | 10 | 98% |
| 70 | 9408.4111 | 589.0335 | 16 | 246 | 6 | 98% |
| 81 | 6223.4322 | 566.7744 | 11 | 414 | 11 | 97% |
| 60 | 8451.7332 | 651.1412 | 13 | 411 | 12 | 97% |
| 17 | 20925.8352 | 1163.554 | 18 | 135 | 4 | 97% |
| 6 | 41661.3307 | 1488.913 | 28 | 64 | 2 | 97% |
| 24 | 8564.8616 | 659.8433 | 13 | 1382 | 53 | 96% |
| 3 | 47079.9018 | 1472.255 | 32 | 94 | 4 | 96% |
| 12 | 10843.05 | 723.8778 | 15 | 1003 | 47 | 96% |
| 85 | 10613.5179 | 625.3324 | 17 | 42 | 2 | 96% |
| 102 | 8578.7192 | 572.9224 | 15 | 41 | 2 | 95% |
| 69 | 6977.0333 | 582.4273 | 12 | 541 | 31 | 95% |
| 40 | 22204.5132 | 1111.234 | 20 | 58 | 4 | 94% |
| 91 | 8400.0822 | 601.0137 | 14 | 158 | 11 | 94% |
| 86 | 11712.0471 | 977.0118 | 12 | 136 | 10 | 93% |
| 103 | 8429.0572 | 562.945 | 15 | 95 | 7 | 93% |
| 72 | 4735.3786 | 592.9301 | 8 | 561 | 46 | 92% |
| 83 | 22852.7966 | 953.2077 | 24 | 94 | 8 | 92% |
| 14 | 15126.0543 | 1164.551 | 13 | 69 | 6 | 92% |
| 44 | 4935.6838 | 706.1055 | 7 | 187 | 17 | 92% |
| 109 | 8326.511 | 556.1086 | 15 | 66 | 6 | 92% |
| 19 | 19800.8506 | 901.0465 | 22 | 513 | 47 | 92% |
| 55 | 9954.1484 | 712.0184 | 14 | 402 | 39 | 91% |
| 78 | 4747.4725 | 594.4419 | 8 | 535 | 57 | 90% |
| 8 | 17923.0206 | 1195.876 | 15 | 247 | 29 | 90% |
| 21 | 11651.0737 | 1060.196 | 11 | 468 | 57 | 89% |
| 76 | 5647.2082 | 565.7286 | 10 | 416 | 51 | 89% |
| 27 | 12275.4213 | 614.7789 | 20 | 121 | 15 | 89% |
| 33 | 14964.8023 | 881.2903 | 17 | 492 | 68 | 88% |
| 25 | 11637.972 | 728.3811 | 16 | 321 | 53 | 86% |
| 79 | 6545.4323 | 596.0471 | 11 | 467 | 77 | 86% |
| 93 | 14550.5862 | 809.3737 | 18 | 48 | 8 | 86% |
| 104 | 6647.9785 | 555.006 | 12 | 141 | 24 | 86% |
| 36 | 13761.2073 | 810.4906 | 17 | 376 | 78 | 83% |

1. A method for imaging or identification of proteoforms from a tissue sample, the method comprising:
spatially surveying a first portion of the tissue sample to obtain survey data with a mass spectrometry apparatus;
selecting from the survey data one or more proteoform targets and a mass-to-charge (m/z) isolation window for targeted fragmentation for each of the one or more proteoform target with a processor;
generating a spatial acquisition sequence for a data acquisition survey of a second portion of the tissue sample with a processor; and
spatially acquiring mass spectrometry data from the second portion of the tissue sample with the mass spectrometry apparatus executing the spatial acquisition sequence.

2. The method of claim 1, wherein the spatial acquisition sequence comprises temporal modulation of a target m/z isolation window corresponding to movement between pixels subdividing the second portion of the tissue sample.

3. The method of claim 2, wherein a space associated with a sample is assigned a proteoform target identifiable within the target m/z isolation window associated with said space.

4. The method of claim 1, wherein the survey data is obtained by proteoform imaging mass spectrometry or wherein the mass spectrometry data is obtained by charge detection mass spectrometry.

5. The method of claim 4, wherein the survey data is obtained by proteoform imaging mass spectrometry and wherein the mass spectrometry data is obtained by charge detection mass spectrometry.

6. The method of claim 5, wherein the charge detection mass spectrometry is individual ion mass spectrometry.

7. The method of claim 1, further comprising identifying one or more proteoforms.

8. The method of claim 1, further comprising identifying one or more proteoforms with a protein or proteoform database.

9. The method of claim 1, wherein a proteoform signature is detected by label-free quantification.

10. The method of claim 1, wherein a proteoform signature is detected by imaging.

11. The method of claim 1, wherein at least one of the one or more proteoform targets has a mass greater than 17 kDa, optionally wherein at least one of the one or more proteoform targets has a mass greater than 20, 25, 30, 35, 40, or 45 kDa.

12. The method of claim 1, wherein the tissue sample comprises a slice of tissue.

13. The method of claim 1, wherein the tissue sample comprises a tumor.

14. The method of claim 13, wherein a tumor-enriched proteoform signature or a stroma-enriched proteoform signature is detected by label-free quantification and/or imaging.

15. The method of claim 1, wherein the second portion of the tissue sample is shifted from the first portion of the tissue sample by less than 200 μm, or optionally by less than 100 μm.

16. A system for imaging or identification of proteoforms from a tissue sample, the system comprising a mass spectrometry apparatus and a processor configured to perform the method according to claim 1.

17. The system of claim 16, wherein the processor comprises instructions for selecting from the survey data one or more proteoform targets and a mass-to-charge (m/z) isolation window for targeted fragmentation for each of the one or more proteoform targets.

18. The system of claim 16, wherein the processor comprises instructions for generating a spatial acquisition sequence for a data acquisition survey of a second portion of the tissue sample.

19. The system of claim 16, wherein the mass spectrometry apparatus is configured for charge detection mass spectrometry.

20. The system of claim 19, wherein the charge detection mass spectrometry is individual ion mass spectrometry.
